SEQUENCE LISTING (10 sequences)

SEQ ID NO: 1 - DNA, 1537 bp, Bacillus amyloliquefaciens
agagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacc

SEQ ID NO: 2 - DNA, 1185 bp, Bacillus amyloliquefaciens
ttagtctccctgaacaataggtgtggtttgaacgatatcttctataacttggtagagaataatcaactttactttaccattctcaacggattggtgcaaaactttttcactttccacctctccgctgccgtttaatcgcttttctacatcttgtcttcccattttgataccggcctgaaccgcttcgcctttcgtataagtccgcacggatgtttcactttctcttgtctgttcttgaacgtaagaaaccggaagcttcatccccagaaattgaaacggatgcttttcttcctctgttttcggatgtttcaacggcttctcattaaattcaaagccccaaaaaggcacgccccatgatccgaaagacagcttgtgcttcgtccttactttacccgtatatagcgtaaatgatgtttcaagaggcacggttacttctgacttgtaccatgtttctccgtaaatttccgcttttgaggcgactttccggtcttcatttccgatcagtcctgagacaaggagctgtcctttgtcaacatgatcgttcacaaaagccatcggctgccccttttgcacgaacatccttttgatgaccgccttctttttggcaacgatatggcgggggctcacgtacttttcttttgtcggttcgtttttttcgaccactttcatatgaagggctgtcccgttcaattccacaccgacccacgtaatattatcgataccgtttgttaatgatttttgaattttctccggcgtcatcattaaaaattgcaaacggccttttttcacaccgatttcttcaaggtgttttaccatctgatgctcggtttctggcttggcccctttaatatcaatttcccaaatcatgttggacaaaagaaacaatataacaaaaaatatcacaaatcccgcagtgaagcccaggtttaattttgatttgagaagcagaaagggaagaccctttctttttgtaaagcttgctttgcattcatgttttctgatgactcttctgaaagcatgtacatcttgaagctgaatcatcagcagaacacagtcttgcttttttttcaccgcaaagatggcgattccgtttctcgtgcattcattcagcagccgttcaattccttttcctttgacttccatttggactttacccgaaaaaaatgacagccatttgtttttcac

SEQ ID NO: 3 - DNA, 1923 bp, Bacillus amyloliquefaciens
atggctatggaacagcagcaaaatagttatgatgagaatcagatacaggtattagaaggtttggaagctgttcgaaagagaccggggatgtacatcggatcaactaacagcaaaggccttcaccacttggtgtgggaaatcgtcgacaacagtattgacgaagccctggccggttattgtacagatattaacatcgagattgaaaaagataacagcattaccgttaaggacaacggacgcggaattccggtcggtatccaggagaagatgggccgccctgcggttgaagtcatcatgaccgttctccacgccggcggtaaatttgacggaagcggatataaagtatccggcggtcttcacggtgtaggggcgtcagtcgtaaacgccctgtcgactattcttgaagttacggttcatcgtgacggaaaaatccattatcaggcgtacgagcgcggtgtacctgtggccgatcttgaagtgatcggtgatactgataagaccggaacgattacgcacttcgttccggatccggaaattttcaaagaaacaaccgtatacgactatgatctgctttcaaaccgtgtccgggaattggccttcctgacaaaaggcgtaaacatcacgattgaagacaaacgtgaaggacaagaacggaaaaacgagtaccactacgaaggcggaatcaaaagctacgttgagtacttaaaccgttccaaagaagtcgttcatgaagagccgatttatatcgaaggcgagaaagacggcataacggttgaagttgcactgcaatacaacgacagctatacaagcaatatttattctttcacaaataatatcaacacatacgaaggcggcacgcacgaagccggatttaaaaccggtctgacccgtgtcataaacgactatgcaagaagaaaagggattttcaaagaaaatgatccgaatttaagcggggatgatgtgagagaagggctgactgccattatttcaattaagcaccctgatccgcaattcgaagggcagacgaaaacgaagctcggcaactccgaagcgagaacgatcactgatacgctgttttcttctgcgctggaaacattccttcttgaaaatccggactcagcccgcaaaatcgttgaaaaaggtttaatggctgcaagagcacggatggcggcgaaaaaagcacgggaattgacccggcgcaaaagtgcgcttgagatttccaatctgccgggcaaactggcggactgttcttctaaagatccgagcatttccgagctgtatatcgtagagggtgactctgcgggcggatcagcgaaacagggacgggaccgtcatttccaagccattctgccgctgcgcggtaagattctgaacgttgagaaagccagacttgataagattctctcaaacaatgaggtcagatcaatgatcacggccctcggaacaggaatcggagaagattttaatcttgaaaaagcgcgttatcataaagtggtcatcatgacggatgccgatgttgacggcgcccacatcagaacccttttattaacgttcttctacagatacatgcgggaaatcatcgaaaatggctatgtctacattgcccagccgccgctttataaagtgcagcagggaaaacgagtggaatacgcttataacgataagcagcttgaggagctgttaaaagagcttccgcaatcacctaagcccggcctccagcgttataaaggtcttggagaaatgaacgcgactcagctttgggaaacgacaatggaccctgcgaccagaacgcttctgcaagtcaatcttgaagatgcaatggacgctgacgagacttttgaaatgctgatgggtgacaaagtagaaccgcggagaaacttcatagaagcaaacgccagatacgtgaaaaatcttgatatttaa

SEQ ID NO: 4 - DNA, 3582 bp, Bacillus amyloliquefaciens
ttgacaggtcaactagttcagtatggacgacaccgccagcgcagaagctatgctcgcattagcgaagtgttagaattaccaaatctcattgaaattcaaacctcttcttatcagtggtttcttgatgagggtcttagagagatgtttcaagacatatcaccaattgaggatttcactggtaacctctctctagagttcattgactacagtttaggagatcctaagtatcccgttgaagagtcaaaagaacgtgatgtgacttactcagctccgctgagagtgaaggttcgtttaattaacaaagaaactggagaggtaaaagaccaggatgtcttcatgggtgatttccctattatgacagataccggtacttttatcatcaacggtgcagaacgtgttatcgtatctcagcttgttcggtctccaagtgtatatttcagtggtaaagtagacaaaaacggtaaaaaaggttttaccgcgactgtcattccaaaccgtggcgcatggttagaatacgaaactgatgcgaaagatgttgtgtatgtccgcattgatcgcacacgtaagttgccggttacggttcttttgcgtgctctcggcttcggttccgaccaagagattctcgatctcattggtgagaacgaatatctccgcaatacactggataaggacaacactgaaaacagtgacaaagcgcttcttgaaatctatgagcgccttcgtcccggagagccgcctacagtagaaaacgcaaaaagcttgctggattcccgtttcttcgatccgaagcgatacgaccttgcgaatgtaggacgctataaaattaataaaaagcttcatatcaagaaccgcctgtttaaccagcgccttgcagaaacactggtggatccggaaaccggtgaaattctcgctgaaaaagggcagattcttgacagaagaacacttgataaagtactgccatacttagaaaatggaatcggcttcagaaaactttatcctaacggcggcgttgtcgaggatgaagtgatgcttcaatccattaaaatctatgctcctaccgatgcagaaggagagcagacgatcaatgtgatcggcaatgcttacatcgaagaagcgattaaaaacattacgcctgctgatattatttcttctatcagctacttcttcaacctcctgcacggagtgggcgacactgatgatatcgaccatctcggaaaccgccgtctgcgttctgtaggtgagctcctgcaaaaccaattccgtatcggtttaagccggatggaacgtgtcgtacgtgaaagaatgtctattcaagacacgaatacaattacgccgcagcagctgattaacatcagacctgttattgcgtctattaaagagttcttcggaagctcacagctttctcaattcatggatcagacgaacccgcttgctgaattgacgcacaaacgccgtctgtcagctctcggaccgggcggtttgacgcgtgagcgtgcaggtatggaagtacgtgacgttcactactctcactatggccgtatgtgtccgattgaaacgcctgagggaccgaacatcggtttgatcaactcattgtcatcatttgcgaaagtaaaccgctttggtttcattgagacgccataccgccgcgttgatcctgaaacaggaaaagtaacgcctagaatcgactacctgactgctgatgaagaggataactatgtcgtagcccaagcgaatgctaagctgagcgatgacggttctttcttggatgacagcatcgtagcgcgtttcagaggggaaaacaccgttgtagcccgcaaccgcgtggattacatggacgtttctcctaaacaggttgtttctgctgcgacagcatgtattccgttcttggaaaacgatgactcgaaccgcgccctcatgggagcgaacatgcagcgtcaggctgtgcctttgatgcagccggaagctccgatcgtcggaacgggtatggaatacgtatccggtaaagactccggtgcagccgttatttgtaaacaccctggtatcgttgaacgggtggaagcgaaaaacgtatgggtgcgccgctatgaagaaattgacggccaaaaagtaaaaggcaacctggataagtacagcttgctgaaatttgtccgctccaaccaaggtacgtgctacaaccagcgtccgatcgtcagtgtcggcgatgaagtagtcaaaggagaaatccttgctgacggaccttcaatggagcttggcgaacttgctctcggccgcaacgtaatggtcggcttcatgacatgggatggttacaactatgaggatgccatcatcatgagtgaacgccttgtgaaagatgatgtatacacatctattcacattgaagaatatgaatcagaagcacgtgatacaaagcttgggccggaagagatcacccgcgatattccaaacgtaggggaagacgcgcttcgcaatcttgatgaccgcggaattatccgtatcggtgcggaagtcaacgacggagaccttctcgtaggtaaagtaacgcctaaaggtgtaactgagcttacggctgaagaacgccttctgcatgcgatctttggagaaaaagcgcgtgaagtccgtgatacttctctccgtgtgcctcacggcggcggcggaattatccacgacgtaaaagtcttcaaccgtgaagacggcgacgaacttcctccgggagtgaaccagcttgtacgcgtatatatcgttcagaaacgtaagatttctgaaggtgataaaatggccggacgtcacggaaacaaaggggttatctcgaagattcttcctgaagaagatatgccttaccttcctgacggcacgccgatcgatatcatgcttaacccgctgggtgtaccatcacgtatgaatatcggtcaggtattagaacttcacatgggtatggctgcccgctacctcggcattcacattgcgtcacctgtatttgacggcgcgcgtgaagaagatgtgtgggaaacacttgaagaagcaggcatgtcaagagacgctaaaacagttctttatgacggccgtacgggagaaccgtttgacaaccgtgtatctgtcggaatcatgtacatgatcaaactggcgcacatggttgatgataaacttcatgcccgttctacaggtccttactcacttgttacgcagcagcctctcggcggtaaagcccaattcggcggacagcgtttcggtgagatggaggtttgggcgcttgaagcttacggcgcagcttacacgcttcaagaaatcctgactgtgaagtccgatgacgtggtcggacgtgtgaaaacatatgaagccatcgtcaaaggcgacaatgttccagagcctggtgttccggaatcattcaaagtattgatcaaagagcttcaaagcttaggtatggacgtaaaaatcctttcaggcgatgaagaagaaatagaaatgagagatctagaagacgaggaagatgcgaaacaagctgacggccttgcattatcaggtgatgaagcgccggaagaaacagcatctccagacgttgaacgtgacgcagtaacgaaagaatag

SEQ ID NO: 5 - DNA, 1635 bp, Bacillus amyloliquefaciens
atggcaaaagatattaagtttagtgaagaagcacgccgcgctatgcttcgcggtgtcgatgcgcttgctgacgctgtaaaagtaactttagggccaaaaggacgcaacgtggttctggagaaaaaattcggttctccgttaattacaaatgacggtgtgacaatcgcaaaagaaattgaacttgaagatgcgtttgaaaacatgggtgcgaagcttgtagctgaagtagccagcaaaacaaacgacgttgccggtgacggtacgacgactgcgactgtacttgcgcaggctatgatccgcgaaggccttaaaaacgtaactgcgggggctaaccctgtcggcgtgcgtaaaggtatggaacaagccgtgactgtagcaatcgaaaacttaaaagaaatttctaagccgatcgaaggcaaagagtctatcgctcaggttgctgcgatctctgctgctgatgaggaagtcggaagccttatcgctgaagcaatggagcgcgtaggaaacgacggcgtcatcacaatcgaagagtctaaaggtttcacaactgagcttgaagttgttgaaggtatgcaattcgaccgcggatatgcgtctccttacatggtgactgactctgataagatggaagcggttcttgacaatccttacatcttaatcacagacaaaaaaatcacaaacattcaagaaatccttcctgtgcttgagcaagttgtacagcaaggcaaaccattgcttctgatcgctgaagatgttgaaggggaagctcttgctacactcgttgtcaacaaacttcgcggcacattcaacgctgttgccgttaaagctcctggcttcggtgaccgccgtaaagcaatgcttgaagacatctctgttcttacaggcggagaagtgatcacagaagacttaggccttgacctgaaatctactgaaatcggacaattgggacgcgcttctaaagttgtggtaacgaaagaaaacacaacaatcgtagaaggcgcaggcgacactgaaaaaatcgctgcacgtgtcaaccaaatccgcgctcaagtggaagaaacaacttctgaattcgacagagaaaaattacaagagcgtcttgcgaaacttgccggcggcgtagctgtcatcaaagtcggcgctgcgactgaaactgagctgaaagagcgtaaacttcgcatcgaagacgccctcaactcaactcgcgcagctgttgaagaaggcatcgtatccggcggtggtacagcgcttgtcaacgtatacaacaaagtcgctgcagtggaagctgaaggcgatgcgcaaacaggtatcaacattgtgcttcgcgcgcttgaagagccgatccgtcaaatcgcgcacaatgcaggccttgaaggatctgtcatcgttgagcgcctgaaaaacgaaaaaatcggcgtaggcttcaacgctgcaaccggcgaatgggtaaacatgatcgaaaaaggtatcgttgacccgacaaaagttacacgttctgctctgcaaaacgcagcttctgtagcagcaatgctcttaacaactgaagctgttgtcgctgacaaaccggaagaaaacgccggcggcgcaggaatgcctgacatgggcggtatgggcggtatgggcggcatgatgtaa

SEQ ID NO: 6 - DNA, 14949 bp, Bacillus amyloliquefaciens
atgaaaagtgatatgaaggcaagcgggttactcaagccatctgtcagctatggaaaatcattgcggctttcagcggatcagccgatgacgattcctgaagtcctgcacaaaaccgccgcggcagcgggagatcaaaagggcatcacatatatacagcctgacggcaccaaagtgtaccagacttacagcggcctgaaaaagatcgctctttcaattgtgaaagggctccgccgatcaggagtgaaggctcaggatgaagtcatattacagctaagtgacaattcccagctgattcctgctttttggggctgtgtttttttaggagcgatcccggttccgcttgcggccgctccggcttatacagaaatgaacagcgggacgcaaaaactgaaagacgcctggatgctgttgaatcggccgtatgtcatcacaagccgtgacgtgctgccggagatgactgaatgggcggaggagcaggagctttccgggttttgcgctttggcggcagaagatctcagtgcgcacgaaatggatacggactgtcatcatccccgtccggaagatttggcgatgctgcttttgacctcgggaagcacgggcacgccgaaagccgtgatgctcagccatgaaaacatcgtctgcatggtcaaaggaaatattcagatgcagggctatacaagtgaagatgtcacgtttaactggatgccttttgaccatgtcggcggcatcggaatgcttcatctgcgtgatgtttatctcggatgtgaggaaatcaacataccgagcgaatccattttgatggaccctttaaaatggctcgacctgattgatcattaccgcgcttcggttacgtgggctcccaattttgcctttggactgcttgccgattttgcagaagagattcaaacgcggaaatgggacttatcttccatgcgttacatgctgaacggcggggaggccgcggttgcaaaagtcggacgcagaattatggagcttttagagccgcacggtctgccggctaatgccatccgacccgcatggggaatgtccgaaacgtcttcaggcgttattttctcagatgagtttacactggaaaacacgagtgacgacgatcgtttcgtcgagatcggtctgccgataccgggatttaatatgagaattacggatgaccgcaatcaagtggtggaagaaggagaaatcggccgcttccaagtctcgggtctgactgtaacgagcggatattatgagcgcccggacttaaatgagagtgtctttacagaagacggctggtttgaaacgggagatctcggatttctgcgtgaaggccgcctgacgataacagggcgcacgaaagatgccatcattattaacggtgtgaattattacagtcatgccattgaatcagccgttgaggaattgccggaaattgaaacgtcttatacagcggcatgcgccgtccggccgaatcaaagcacgacagatgagctggcgattttctttgtgacatctgttccgttggatgagaaccgcatgacgaagcttcttcatcacattcaccagcacgtgacacagagaatcggcgttacccctgattatctgcttccggtcgcaaaagaagacattcctaagacggcgatcgggaaaatccagcgcactcagctgaaacattcattcgaacaagggcaatttgattcattacataatagcaggcaagaaggcggcaattcagaaaccgcttctgccgagaaagatattgaacgggattttatccgttttctgaaagagaaactttccatcgcggatgagctcgttgatccaaatacgccgtttcaaagtcttggcgtcaattccatcaagatgatgaaactcgccagatcaattgaaaaaacgtatcacatccggctgacggcaagagagctgcacaaaaacccgacgattggcgctttagcggcttatactgctgaaaaagcggcaaacatgtcagcggagcatcatccccaaaaagccgaaccgccggcagaacgggagagacggcaaacggcgccagcgctttcagaagtccaaaaagggctgtggacattgcagaaaatgtcgcctgaaacgactgcataccatgtcccgctctgtttccgattcacatccggaatcgacaaagagaaactgaaacaggcgtttcagctcgtattaatgcagcatccgatgctcaaaagcgccgtgaatgaaacagacggcgggttatatttcatagaagcggcagatccttttgtattctcggaagaagatatttctgacgtcaatgaatcacaaatatccgctctgatacggaaaaaagtgaaagagccgtttgtcaaagaaggcgggcctttgctgcgcgtgcagctgctttcaaaatccgccgaggaacattatcttttgacggtcattcaccacatcgtgtttgacggcatttcatccattacatttattcacagtctgctggatcattatcaggcgctgctggagggaaaagaagagtttatggccgccgctcccggcattcatcccgatttcgcggcttgggaaaagcggtatttggcgggtgaagaaagcaaagctgcccgcgcttactggatgaaacagctcgggggcgatctgcctgaccttcagcttccgccgacgcgcacggacagcagtgttccggagtttacagaagatacgctgacacgccgtcttcccgaacggctgctgaataatataacggcgtttgccgcatcccgttcgattcatttgtctaccgtcatgctgagctgttacatggtgcttttaagcaaatatacggataaggaagatattgtgacaggcatgcctgcaatggtcagacccgaagaacgctttgacggagtggtcgggcatttcctcaacatgcttcccatacggagcagagccggacgaaaagaaacatttgctgactttgttcaaaaggtgcaggatgccgttctggacggactggatcactcattttatccgtttccgaaaatggtgcgtgatgtgcatgcgaacccccggcagaacggttcgcctgttttcagcaatgcctttttctatcagaactttctgcagcattcaagctatcaatcgatgcttgatgaatacaaggaattctcctgtgagtttgtaaaagacattcatcaggagggggaatatgatcttgtatttgaaatgtgggaagaagcatcaggcatggatcttacgattaaatacagcaccgaattattcgacgaagcgggggcggccagacttttcagccaatttgtacaggctgctgaagctttgacggcgaatccggatacgccgcttgagaacgtttctctcatgacaaaaagagaggagcacatgattcttaagacttggaatgaaacaagccgtgaatatccggatgcctgcttccatgaattattcgaacgtcaggcggcagaaacgccggacgcttgtgcggttgtgtatgaacagcaacagctgacgtaccgtgagcttgatgaaaaaagcacgaagctggcgctctatttgcaggcgcacggtgcaggccctgatgatctcatcggcatttatacagaccgatcattgcatatggctgtcggacttctcggcattttaaaagccgggggagcgtatgtgccgcttgatccttcttatcccgctgacagattggaatatatgatcgccgacagcaggatttcaatgtgcctgacgacagctgatttagagcactctttgaactggggaggcgtgcagaccactgccattgaccgcgattggtctcatattgaaagtacagccgcggaaagaacgtcgctgaagcggttagtgacacctgacgatctggcgtatgtcatttacacgtcgggcagcacgggcaaaccgaaaggcgtcatgattccgcaccgggcactgaccaatttcttactctcgatggcgaatgaaccggggctgtcgtccgaagacaaactgcttgccgtcacgacatattgctttgacatcgcggcactggagctttaccttcctttaatcaaaggagcggagtgcaacatctgtaagacagaagtcacaaaagacgcacggaagctgaaagagctgattcaagagtacaaaccgacgatcatgcaggcgacgccgtttacgtggaaaatgctctttcattccggatggtcaaatgaagaaaaagtgaaaatcctttgcggcggagaggcgcttagcgaacaattgaagcagcagtttctcgatacgaaaagcgaggcttggaatatgttcgggccgacagagacgacaatctggtccgcggtgcagcggattacggaaaatgagagcgcgcttacgatcgggcgcccgatcgccaatacccgcgtgtatattatggattccggtctcaaccctgttttagaaggggtgccgggagaactttgtattgcgggagacggattggcaagagggtatttcaacaagcctgagctgacagataaagctttcgtcagccaccggctcgaactgggatcaaagctttataaaacaggcgatatggcgcgttttcttcccggcggccgcattgaatacatgggacggatggatacccaagtcaaaatccgcggataccgcattgaacccggagatattgagagcaggctgaatgcccatccggccgtacaggaaagtgtagttgttgtgaacaatcactccggaaatgaaaagctttgcgcgttttatatccggaaaaacggagagccgcttccgtctgcaaaggaattgcgcaatcatttgaagcaagcgctgccggcctatatgacgccagcttcgtttatccgtcttgaagagctgccgctcacaccgaacggaaaagtggaccgcaagcttcttgctgcccgcgatctgacggaaaagcagccggacacacaaacattttcatcatctcatatccagcaggcggtactggcgatttggcaggatgtattgaaaagcaaagatatagagcttgatgaccgcttttttgatgcggggggagattcgcttcttgccgtgactgtcactgaccggatgacgcaggaactggattgtgaattcagtgtaacggagctgtttgaatacgcgacggtaaaagacatcagccggtatatcgctgaacaaaaaccgaaagaagccgtttgggctcctgtaccgcaaaagaaagtgaatgcagaagaccgggaacatccggcgggagaatttcccgattattacgaggacagccttgcggttatcggtatatcctgtgagtttccgggagcgaaagatcattatgaattttggaataacatcaaagaaggcaaagaaagcatcaacttcttttcaaaagaagagctccgccgattcggtatttctgaagaactggctgatcatcccggcttcgttccggctaaatcagttatagaagggaaagaaatgttcgatcccggtttcttcggcttttcaccgaaagatgcggaatatatggatcctcagctgagaatgcttttgctccattcatggaaggcaatcgaggatgctgggtatatctccaaagaaattccggaaacaagcgtgtatatgtctgccagcacgaactcttaccgcagcctgctgccggaggagacgacagcacagctggaaacgccggatggctacgtgtcatgggtgcttgcccaaagcggtacgattccgacgatgatttctcataaactcgggttgaaaggaccgagctattttgtccatgccaactgctcatcttcgttaatcggcctgcattcggcttttcaaagcctgcagtccggcgaagccaagtacgcccttgtcggcggtgcgacccttcatacggaatcaagcgccggatacgttcatcagccggggctgaatttttcaagcgacgggcatattaaagcgtttgacgcggatgctgacggaatgatcggcggtgagggagccggcgccgtgctcttgaaaaaagcgtctgatgccgttaaggacggcgaccatatttatgcgctgctgcgcggaatcggcgtcaataacgacggagcggataaggtcggattttatgcgccgagcgtcaaaggacaagctgaggtcattcagaaggtcattgatcagacgggcatccatcccgaaacgatcgcgtacgtcgaagctcacggcacaggaacaaaactcggcgatccgatcgaactgtccgcacttcaatcggtgtacggccgatatacagataaaaaacaatactgcggcatcggttcagtcaaaacaaatctcgggcatttggatacagcggccggaatggcgggctgcatcaaggttgtcatgagcttatatcatcaggagatcgcgccgagcatcaattacaaagagccgaacccgaatcttcatttagaagactctccgttttacgtagcggaagaaaagaaagagttaacccgtgagaacagagcgcaccgcatggctttaagctctttcggtcttggcggaacaaacacacacgcgatttttgaacaatatccggatgcatccgaagcagcagacgctgccggtccattcatcattccgttgtctgccagaaaaaaagatcgcctgaaggagtacgcaaaacagctgctggcttttctggaaagaaaaacggacgccgacctcgctgatcttgcctacacgttccaagtcggaagggaagcgatggaagagcgcgcggcctttatcacaagcggcactgcggagctgaagcggcagcttgccgattttatcaatgataagccggctgtaacaggctgcttcagaggagaaaaacagcaggcgaaagatattgcctggctttctgatgatgatgacagcgctgaactcattgagaaatggcttgcgaaaggaaaaggatcgaagctctgtgaaatgtggagtaaaggtgtcgccatcaattggcacaagctttacaaagacaagcatccgaaacgaatcagcgtgccggtctacccgtttgcaaaggagccgtactggccgaaaaaagcagaaaagaacacatctgccgcacacaccggagtctcagttttacacccgctcgtgcagcaaaatacgtcagatttaaccattcagaagttcagctcactgtttacaggatcagaatttttcctgaaggatcatgtggtcagggaaaaagcggtgctgccgggagcggcatatttggaaatgtcttatgaagcggtaaaacgggcgcttggcggcttattaagagaccaagacagaataacgctccatcataccatctggatgaaaccgatcgtcgttcacgaacaagaacgccgtgttcatattgcgctgttccctgaagaggacggcatcatctcatatgacatttacagcgtaaacgctgacgatgaagaagtgctgcacagccaaggacgggccgaaatcataaaggctgaacaagatcctgagacagacctttctgccatacaaaacagatgcacagcggacattcttgatccggcggccttttatcgggaaggccggagcagggggatgttccacggaaaagccttccaaggcattaagtccgcctttatcggcgagaaagaagtgctttcggacatacagcttccggattctgtttctcacacgaacggacaatttacgcttcatccgagcatcattgattcagctattcagacggcgacgatctgcattatgcaggagttttccggacaaaagctgattctgccgtttgcactggaatatcttgaagtcgtcaaagcatgcacaccgactatgcgggcgcatgcacgcttcagtgaaggatatcaaactggtgattccgtgcagaaagccgacattgacctgttcgacgaaacaggagcgctttgcgtgaaggtgagaggattctctacgagagtgcttgaaggcgatgcagaatgggctcagcggctatcaggctcggaaaaggtcttgtttgcaccggtctggaaagaagccgccgcatccgagaaagcctctgtccatgctgatgagcatttgatcatactttgtgaagatgcctgccgtataaaagatgaagcggcatccggcctgaaagacgccgaactgctgattgcagaaggtgagggtgaaacagccgctgaacgcttccaaagctttgcgcagacaatcgctgaacacatccggcggatcatcagcgaaaaaaggcagggccgcattctgattcagactgttatttcagcagacggcagccagcaattgtttgcggggttaacaggtttgttaaaaacagccgagctggaatacggaaaacttgactgtcagctcatcgaaatggaatcaatcggcgaaatgccggctgtcgctcaaaaactgaaaaatgacagtcatcggccgcatgataaacatatccgttataaaggagaaaaacggtatgtgaaggcttgggaagaaatgcatcccgcagggggcggcaccgtctggaaggacgaaggcgtatatctgattaccggaggggcaggaagcctggggttgatgttcgcaaaagaaatcgcaggtcgcgtaaaacacccgaaaatcattctgaccggccgctcaaatctgccagaagccaaacagcgtgagcttgctatgcttcagcactcaggcgcaaatgtcgtctacaaaaagctgaatgcggcagaccggcgggaaaatgaagcgctgataaaagagattacggcggaattcggaaagcttaacggaattattcacgctgcgggagtgatacaagaccgcttcctgctgcaaaaaacgaaggaagagttcagggaagtgcttgatccgaaagtcatcggaaccgcttatttagatgaagccagcaaagactgcaatcttgactttttcgtattgttctccagtgtttccggcgttctcggcaatgtcgggcaggctgattatgccgccggcaacgcgtttatggatgcatacgccgcttacaggcaatcccttacggagaatggccttcggcacggaaaaacggtcgcctttaactggacgctgtggaaagatggcggtatgcaggcgggagacgagacggagagaatgatgaaaaaagctttcggcatggttccgcttcaaaaggcgtcaggcctggctgcattttatgaaggaatggctcaggaaaagccgcagcttttagcagccgaaggacacgcagcaaaattaaaacagtcgttcctatccgtttctgaaactgaaaagccgcaggtggagaaaattgagcctgtatccgccgtttcgggagacaaatggcacggtgcgcttatccgattggtcagttcaattttaaaagtcggccaagacgagattgatattgatacggaactgagtgaatatggttttgattccgtatcgtttaccgtctttacgaatcaattaaatgaagcgtatcagctggaactggcgccgacgatcttcttcgaacacgggacgatccgcagtttagcagagtatttgacggatgaggcagaagccggccttccttctcagccggaagaaaaacacgatgcagaagaatctcttcaaacgcttcagaccgctttgacagccatggtaagcggcattttaaaagtagaccgtgaggacattgagacagatacggaattgagtgaatacggttttgattccgtatcgtttaccgtctttacgaatcaattaaatgaagcgtatcagctggagctggcgccgacgattttctttgagtacggaacaatcagcggtctcgccggatatttggcaaaagagcatccgggccatttcggcgaaaagaagaaagagtctccggaaaaagcgcagccaaaagcccaaaaaccgaaaatgcagcggaaaaagcgcttcgcaacggtaatgaacgctgcggcagcgacacaagagccgcgcggcaatgacccggttgccattgtcggaatcagcggccgattcccgggtgcaaaggatatagaggaattttggcgcaatttgaaagaaggaaaggactcgatcacaaccattccgaaagaacgctgggattggcaagcgtttgatggcgacccgaatcttgaagggaacaaaacgaatatcaagtggggcggctttatcgacggaatagctgaatttgatccgctgttctttgggatttcaccgcgggaagcccaatatttagatccgcagcagcggctgttattgacttatgcatggcgggcgatagaagatgcgggctgtaagccggaaagcctttccggcacaaatacgggcgtctttatcggcaccggaaataccggttataaagatttgttcacaagggtaggtctagcgccggaaggacatgcggcgacaggcagcatgattccttccataggtccgaaccgtttgagctatttgttaaatcttcacgggccgagcgagccgattgaaacggcatgttcaagctcgcttgtggcgattcaccgggcggtatccgctatagaaaacggagaatgcgacatggcgattgccgggggaatcaatacgattctgacggaagaagcgcatatcagctacagcaaagcaggcatgctcagcaaagacggaaaatgcaaaaccttctccaaagatgccaacgggtatgtcagaggagaaggcgcgggaattctgatgctgaagaaactgacagatgcagaacgtgacggcaatccgatttacggcgtgatccgcggtacggctgaaaaccacggcggccgggcgaatacgttaacctcgccgaatccgaaactgcaggctgatttattggtgaaagcataccggaaagcgggagtggacccgagcaccgtcacgtatattgaagcgcacggtacaggaacggaattgggcgatccgattgaaataaacggtttgaaagcggcgtttcacgagctggcgaaaacaaatcaagagcccgaagtctccggtcaccgctgcggcatcggttctgtcaaaagcaatatcggccatttagagcttgccgcgggggtatcaggcgtgatgaaagtgcttttgcaaatgaaacacaaaacgctggtgaaaagcctgcattgcgagacgatcaatccgtacattcagcttgatgacagtcctttctacatcgtgcgggaaaatcaggaatggacggcagcaaaagatagaaacggaaatgccattccgcgccgggccggggtcagctcatttggcatcggcggtgtcaacgcccatatcatcattgaagagtatgtcccgaaagcagccgctcagcctgagcacagcccggaaaatccggcaatcatccttttgtccgccaaatcaaaagaaaaactgtttgaacaggcgcagcagctgcaaaaagcgatccgtcaaacgccgtatacagatcaagacttggcgggcgtcgcttatacgctgcaaaccggaagggacgaaatggaagagcgcttggccattcttgcagcgacgatggcggaattggaaacaaagctcgaagcatttacgaaaaatgagaaaaacatcgccgggctgtataccggccagtctcaccgcaataaagatacgtttgccttgttcacggcagatgaagatatggacatcgtcattgacgcctggatcagaaaaggaaaattcgccaagctggcagaggtatgggtgaaaggcggcgtattgaattggaaccggctgtatgaaaagggtacgccgcgtttactcagtcttccgtcatatccgttcgcaaaagacacgtattgggtgcctgagaatcctgacaaaaataaggtccatacagaggaacggatgaaacgtattttgacgaaacaatgggaactgagcccgctcgaactgcgtgaaccgtgcgcacgttccgttgcgattttgacaggcggggacacgatcaatctcgcagaagagatcgcagcgcatatgccgaatcaccgtatcatcacagcatcagaccttaccggtgaatatgactggcaggcgtttgacggagtcatcgacctcctcggcgcagcgaaagaagattctcaagacatggccggaatcaaatggcttcagcaactgattgagcatgggcataaagaaggggtgacactgctttgtgtcacgaaagaccttgagtcattaaataaagaggccaatcaaacaacaggagcaaaacgggccggtctgtacagaatgctggcatacgagtacggtcacctgtcttcaaggcatctcgaccttgaaggagcgctttcggatgacaaactggcgaagcagattgccgatgagtttcatgcccgcactgatgacgcggaagtttgccggagggacggcttgcgttaccgcgctgtattaagcggctcagaaaacatggatatgaaaggtgagagaattgattttccggacaaccatgtgctactgatcaccggggggacgagggggatcggtcttttatgcgcacgccattttgccgaacattacggagtgaaaaaactggtgcttacaggccgtgagacccttccgccgcgaagtgagtggacgggcggactgaatggggtgccggcttctgtaaaagcgaaaattgaagctgttctcgacttggaatccaaaggtgtacaagtgaaggttttgtcagtcccgttagctgatgaagccgggctgagacaggaactgagtcagataaaacagacgctcggacctatcggcggcgtgattcactgcgcgggtgtgaccgataaagagacgctcgcattcatcagaaaaaccgatgaagacatacagcgcgtgctcgagccgaaagtagacgggcttcaggcgttatacagtgtgctcagcgaagagccgctgaagtttttcgtcttgttctcttccgtgtcagctgcgataccggcgctttcggcaggacaggccgactatgccatggccaatgcgtatatggactattttgcaaactcatacaaagagcagctgccgatcgtaagcattcagtggccgaactggaaagaaaccggaatgggcgaagtaacgaacaaagcttataaagagagcgggctatacagcatcacgaatgcagaaggattacagctccttgacggcatacttcttgaatcagcaaggggcccggtgctgccggccgttctgaatcctaagctgtggaagccggagcgattcctgcggcggagcaggcaggatcagccaatgagtccggctatggtaaaaccggcggaaaaaaccgcttacactgccgcagacaacgctcaattaacaaagaaaacagaagaatggctgaaagaattattttctgaagagctgcggattgaccaggaccagcttgagacagacgtattattccaagattacggagtggattccattattcttgctcagcttttgcagcggatcaatcggaatctctcggcatcgcttgacccgtccattctgtatgaatatccgacgattcaatctttcgcaaattggcttctcgaaggatatacagaagttttgtcggaacggtttgatttggcggcggagtctgttaagaagaagccggctgagccggtgattgaaacacaagccgttaaggtgcaagaagaggcggccggaaaacagattcaccgggaagatatcgcggtcgtcggcatgtcctgccggtttccgggggcggcttccttagaggcgtactggtcgcttttggctgaaggccgttcggctatcggtcctgttccggctgagcgatgggggttgaagacgccttattacgcgggaatgcttgatggaattcatcaatttgatccggacttctttttactggcagaagaggatgtaaaggcaatggaccctcaagctttggccgctctggaagaatgccttaacctgtggtatcacgctggctatacgccggatgagataaagggagaagcaataggtgtgtatctcggcggacgcagccgtcacagaccgggtgaagagaaactgctggatgcgaaaaacccgatcgtcgcgctgggtcagaattatttggccgcaaatctctctcaatattttgatatgcgcggcccgagcgttgtcctcgatacggcttgttcttctgctttagtcggaatgaatatggccgtgcaggctcttgtaacaggagaaataaaagcggccgtcgtcggcggggtgagcctgtttgaatcagaagaaacccataaattatttgaacagcgcggcatattaagtaaagcgcagtcgttccatgtatttgacgaacgcgccgatggagtcgtgctgggcgagggtgtcggaatggttcttttaaaaaccgtgtctcaggcgattgaagatggagattctatttatgccgtagtgaaggcggcatccgtcaataatgacggaagaacggcaggcccggctacgccgagtctggatgcgcaaaaatccgtcatgaagacggctcttgaaaaaagcggaaaacagccggaagacattactcatattgaagcgaacggttcaggcacggtcgtgacggatctgttagaactgaaggccattcaatccgtctaccgttcaaaagacgctggcccgctcggaatcggatctgtgaaaccgaatatcgggcacccgctttgcgcggaaggaattgcaagtttcataaaagttgtgcttatgctgaaagaaaagagctttatcccgtttttatcaggcgagcatgaaaacacacattttgatagagaaaaggccaatattcagttcagccgcactctggcagactggccgtcaccaatgccggcggcaggaattaactgctttgccgacggcggaacaaatgcccacgtcattgtggaagcgtggcaggaagacgaagggcgccgaatcaagcgccatcctttaacaccgcctgcattaaataagcggctgatttcgccggacagcaaagaagaaacgaacgaaaaagctgcgacaaacatctgggatacctatgaagtggaggtttaa

SEQ ID NO: 7 - DNA, 13425 bp, Bacillus amyloliquefaciens
gtgaagaacttcaataagcaaatcactttatctctgaaaaatccgtttattttacatcatgacgtttacggacaaaaagtccttccgggattggcttatattgatatgttttatcagattttcaaagagcgcggctattcatacgacgggcttgagctgcgccatttatccatatatcagccgcttattgcgaaagaagacagcgccgtcaagcttacggttgaatgtacaaaggtaaaagaagggcgctggaaacttaccgcaaaaggggctgaggtcagcggcggacgagtccagccggaagaacgcatttacgtcaaagcggaaatgcacgagacggcacccgcagttttcagtgacacgcttgatcttgactctgtcaaacgcgaggcggaacgcaccatcaatttaaatgagctgtatgcacaatgcagaagccaggaattggttcacagcggatatatgaaagcagaaggagaagtgtttgaggagcgggacggcgtcacctttgatgtatcgctcggaaaagacgcgatgggacaggccggagggtttttgttccatccgactcttcttgacggaagcggcgtcggcgcaaacctattgatctcatcgttgttaaatggggaaaagaggctgtttctgcctcttttctacgaatcttttacagcttctgaacctttgcaggccggctgcatcacgagaatcaaacgttcgtcgcttcggcaggaaaaggagcttatttacattacactggagttttttaacgcatccggccggaaagtagccgagctgaaagatttcacaagcaagctcgtgcgggatgccgagctgattaatacagaccgtaagtctgctgagaggtctcctgaaaaagccggtttaccggaagttgcggcatcgcttgaaggcggacacccgcatgaggaagcagaacggtatgtcatgaatctgatggcggaaaaaatcggcaagccgcttgaacagattgattctcaagtcggctactatgaaatggggctgacatcgtccggactgcttgacgtcgtcgaaacgatcagcgaaaaaatcggcgaaaccctttctccgactttgctgtttgaatatacgacggcagcagagttggcggctttcctgactgagcagtacgggagccatttcagcgcgtctccggcaaaaatggagaagccggttgagacgcaaaccgaaaaaaggcggaaaagcagtgtgccgcaagatgaagatatcgccattatcggcatggccggccgctacccgcaggccgaaaatattcatgaattctgggaaaacctgaaagaaggaaaaaactgtgtcaccgaaattccggagtctcgatgggactggcgccggtttgaaaatgtaaagtcgccttccggaaagagcatttcaaaatggggcggatttattgatgatccggattgctttgatccccaatttttccggattacgccgcgggaagctgaaacaatggaccctcaagaacggctgtttttacaaacatgctgggaaacgattgaggatgccggatatacgccgaagtcgctcgcagaggcgcgcggacggaataagagacagcgcgtcggcgtcttcgcaggggtgatgcataaggattatacgttggtcggggctgaagcttcttctgaagatcatgtattcccgctttctcttaattatgcgcaaatcgcaaacagagtttcatatttttgcaattttcacgggccgagcatggcggtcgatacggtctgttcatcttcgctgactgcggttcatcttgctgcggaaagcattcggcgcggggaatgtgaatcggcggttgccggaggcgtcaacttgtcgctgcatccgaataaatatctgacatacggtttatgggacatgttttcaacagacggggtttgtcatacattcgggaaagacggagacggctatgtaccggctgaaggaatcggtgccgttttattaaaaccgctgcgagaggcggtaaaagacggagaccggatttatgcggtgataaagggcagtgccgtcaatcacgtggggacagtgagcggaatttccgtgccgagtcctgtcgcgcaggccgatttgattgaagaatgcctggacaaagcgggcatcgatccgcgcacggtcagttatattgaggcgcacggaacgggaacttctctcggcgatccgattgaggttcaaggccttgtcaaggcgttccgccagtacacacaggacaaacagttctgttcgatcggatcagtgaagtccaatatcggccatgcggaatcggcggcgggcatcagcggcttaagcaaagttgccctgcagcttcaccataaaactttggtgccgtctcttcattccgaggagctgaatccgtatttagattttgaacaatcaccgttttacgtccagcatcataccgaagagtggcagcagccctcagtgacggaaaacggaaatgagaccgtatatccccggcgtgctgggatcagttctttcggggcgacaggttcaaatgtgcatctcattctggaagaatttattccggaagaatcactcggaacaaatgctgaaccggcaggagaacagacattcatttttccggtttcagcccggaatgaagaacggctaaaagagtacgttgcgaagctgtctgcatttattcatcccggcctgccgcttgaggatgcggcatatacacttcagatcggccgtgaacagatggaggagcgggttgcgttcaccgctcagaatatgacacagctcaaagagcggctggaaggctttctggcaggtaaaacggtcagcgggtgctggcggaaccgagctgataaaacgaaacaggcagcgcttgctccagacttgatacataaatggctttcttccggaagtctggagatgattgccgaaaagtgggccggcggaggaacaatggattggaatctcttatacggtgaaaacaaaccgaatcggatcagcctgccgacatatccgtttgaaaaaacacggtattgggtgcccgaggcggagaaaaaagattcgttaccgcaaaaaaatgcaggcatgctgcatccgctcctgcatgaaaatgtatcaaacatgtcaggcgtccgttttcggtctgcgtttagcggcggagaatttttcctgcaagatcacgtcgtcaaaaacgagcggacgttaccgggcgcggcttatctggaaatggttcgggcggccgttgaacacgcggcagaaacgaaggaagaaggaacagtccgtctgaaacatgtcgtatggatacgtccgtttacagcggcggatgagtccgcagaactgtatctccgtctttatgacgaagaaaatggggagatgggatttacggtttacagtgaatcggcttcctcggaactagagcccgttctctatgcgcaaggaagcgcggcgtttgtccgagcgggcgaaccggccaaaatcgagattgacaaattaagagagtcacgccgtctgatcacacctgacgcttgctatcaggcatacaaacaaatggggattgaatacggggatgctcacaaagcggtcacggaaatctatgcaaaagacagcaaagtgctcgcgaagctctcactcccgtcatctatttctcatacaaaagatcagtttgtcctccatccgagccttttggatgcggccttccacgcgacaatcgggttaatgcttgacagagacgggactgacatacggccgatgctgccgtttgctctcgaacagattgatgtgttgaaaagctgtaaaagcgatatgtgggtatccattgcgtacagcggcgaaagcaagcctgaagatgccgtgcagaagctggatattgatgtatgtgacgaacaaggcaatgtatgtgtgaaaataactggttattccgcacgggaaattgaaacggataaaacagatacactatattttgaacacgtatgggaagagaaggccgctgtacatgaaaaagcgccgatccatttcaaagcccgtcacgtgatgatatctgaccggtttacggatcaaatgaacagcatacagacagacttcagctgtcatgtgctccgtgatcagcataagagtgaagacgaacggtttgaggcttgtgcgcttcaagcgttcgaagaagtgaagaaaatcctcgcttcaaattccggcgggcgcgagctcattcagattgtcgtgcctcaagaagcggatacgcagctcctttgcggcttaacggggcttttgaaaacagcggcgcttgaaaacccgaacctgaccggccagctgattgaattggagccggacgtcaaggcagacacgctgtcacgatatgtaaaagaatgcagcgctcatcctgatgacaagcatattcgctatgaaaacggcaggcgctatacggcacaatggcaggaaacaggacttgaacagttcggcgccaaaaagccgtggaaggataaaggtgtttatctgatcacaggcggtgcaggcggtctcggatttttgttcgccaaagaaatcgcccggcatgcaaaagacgccgttcttattttaacggggcgttctctgttggataaccgtaaaaaagcccagcttcaagaactgtccgatctcggcgtgcaagccgtgtacaaacaaacggacatgatggaaaaacaagaagttcaatcgctcacagcggacatctgccggcagtacggcggattaaacggtatcatacacagcgccggaacgataaaagacaactatattctgaaaaagcacaaagatgaaatccgtgatgtactggcgccgaaagtctccggactggttcatcttgatgaagcggcaaaggacattgagcttgattttctgattcttttctcttccggagccggaagcgccggaagcgcgggccaggctgattatgcgatggccaatgcgtttatgaacgccttcagtgaaaaaagaaaccgtaaaaaagcccgaaatgaacgctatggacacaccctttcaatctgctggccgctttggaaagacggcggcatgacggttgatgctgaaacggaagccatgctgacaaaagaaagcggcatggtccctatgcaaacagaaaacggcattcatgctttgtaccgcgggtgggcggcaaacacatcccaagtcatggtgacggaaggctacgccgcaaaaatccgggcgtttttgaaaaaatcagcggatgatcagccggataagggcagcgaaaccgaagaaacggcaggtcttacagcgggcggagagaacatgcttgcagaaaaaacggagcatttcttgaaacaagtgctttcggaagtgaccaaacttcctgtcggcaaaattgacgccaaggcgcctatggaagactacggtattgattcgattatgattatgcatttgacgggccggctggaaaagagcttcggttcattgtcaaaaacattgtttttcgaatatcagaatatcgaatcgctggcgcagtattttatgaaagcacaccgggacacgctgctggaagtgaccgggatgaaaacagaagaaacccctgcagccaaaccggcgctgtctgagaaagcgaaagtaaagcctgttttcaaacggaatgaaggcaaaaaaggctttgcgcaagtccgtccagacacgcggaaaaatcagcagctgacagagcgtgaagatatcgcgattatcggtatatcaggacgatatccgcaggccgaaaatcttcaggaattttggaagaacttgagtgaaggcactgactgcataaccgaaattccaaacgatcgctgggatcacagtctttattacgacgctgataaagataaagaaggcaagacgtacggaaaatggggaggatttttgaaagacgtggacaaatttgatccgcaattcttctctatttcgccccgggatgcaaaattgatggacccgcaggagcggttattcctgcaatgtgtctacgaaacgatggaagatgccggttacacgcgcaaaaagctcacggaaaaatcgggtgatttgcttggcgccaatgtcggtgtttacgtcggcgtcatgtatgaggaatatcagctttacggagcagaagaacaggcgcgcggaaaatcgcttgcgctgaccggaaatccgtcatcaattgcgaacagggcttcctatgtattcggttttaacggcccgagcatggctctggatacgatgtgctcttcttctttaacagccattcatctggcatgccaaagcttgcggaacggggaatgcgaagcagcatttgccgggggcgtcaatgtatccgttcatccgaataaatatttgatgctcggccagaacagatttttatcaagcaaaggcagatgtgaaagtttcggagaaggcggagacggttatgtgcccggtgaaggagtgggagccgtactccttaagccgctttctaaagcgaaagcggacggtgatcatatttacggcctgatcaaaggaacggccgtgaaccatgatggaaaaacaaacgggtattccgtaccgaatccgaacgcccaagccgcggtgatcaaacaggcgttaaaagacgcgggaaccgatccgagggcggtcagctacatcgaagcccacggcaccggaacttcactcggcgatccgattgaaatcacgggtctgacaaaagccttttcagaacaaacacaagataaacagttttgtgccatcggctcagcgaaatccaatatcgggcactgtgaaagcgcggcgggcatcgcgggtctgacgaaggtgctcctgcaaatgaaacataagcagctcgcaccgtcactgcattcccggacgcttaacccgaatattgattttctcgctacgccgtttaaagtgcagcagacgcttgaagagtggaaaagaccggtcataaatgaaaacggaataattaaagaattgccgagaaccgcggggctttcctcattcggagccggcggtgtcaacgctcatatcgtcatcgaagaatattcagctgatgaggataaggagacggcttttgccgcgcctcatccgtcgatgatcgtcctttccgcgaaaaatgagaaacggcttcaaaaacgggcggaacgtcttcttgatgcgctgcggagcgggcgttaccgggaagcggatctgagccggatcgcatatacgcttcaaatcggccgtgaaccgatggaagagcgtctcggaatgattgtcagcaatttgcgcgagcttgaagaaaagcttgacgaatttacgggcggcaaggagtcaattgaccagctctaccgcggacaagtcaaacagaataaagacacgatggcgctgtttgcggctgatgaggacatggaaaaaaccattgtggcgtggcttgaaaaaggcaaagccgcaaaggtattagagctttgggtgaagggtcttccgctgaattgggataagctgtatcaaatgggcagaccgcaaaaaatcagtctcccggcctacccgtttgcaaaagaccgttattggatcgacatatcagcagatgcagctgtcaaaaaaccggccgacacacagtctgcgccgatcgcaccggcggccgtcccggaaccggtcattgaagtaacaactcttcacgaggagactgccaaagaaaaaccgtcaaacatttcattgcagcctttgagcggcgcgaaaccgaaaagaaccgtacagtctgtttcccgtccggcggtttccgttgaaaaagtaagtgccgtattgaccgaaagtttggcagacgttttatatatggatgccgacgatattgacgcagatgacacatttattgatatcggcatggattcaattaccggtcttgaatggataaaggccgttaataaagcgtacggcacgtcccttacggtgacgaaagtatatgattatccgacaatccgccagtttgcggccttcttgcaaaaagagctcggaacagaaccgatctttgaggaagcagccgaaccgcagaaaacagaggccgtgaaaaaaccgtcaaacataatgctgcagcctttaaacgctcataagcagcatgtgatacaagcagaagcggatattctggaggcgatgtctgaaacggcagcttccgccgaaccggaagaaccgattgaagcggtgcatgacgagttatcaaaagcactggcagatgtattgtacatggagcggcatgaagtagatattgatgaggcctttattgacttgggtatggattcgattaccggtcttgaatggataaaggccgtcaataaacggtacggcacggactgtaacgtaacgaaagtgtatgattatccgacaatccgccaatttgccgattttctgcgtacgcagccgtccgtccgcggcagaaaaaaacaggcggttcccgtacgtccaaagccgcttacgcagcaacatgccctgcaggagaaaacacctgccgagcctgagagattaacgcttcaaaaggaacctgaaccggtgcaggccaaacgggagcctaaagatgagatagcggacgcgatagccatcgtcggcatgtcgggcaagtacccggatgcgccggacctaaagacgtattgggacaacttagcccgtgcgaaaaacgccattcgcgacatcccgctttcccgctgggatgtaaacaaatactatgatccggccttaaacaagaagggcaaagtatactgcagatcaatcggtatgcttgacggcattgaagagtttgatccgctgttctttaatatatctccgtctgaagcggaacttatggacccgcagcaccggatatttttacaggaaggttttaaagcctttgaagatgcgggttacagctcaaaagaattgaacgggaaaaattgcggcgtttatttggggattatgaacaatgaatacggcatgatgttaaacaaacatcagacaggaggaagcgcgacgggcaacagtttcagcatcgcggcggcccgcctgccttattatctcaatttgaaagggccggccattccgattgatacggcatgttcttcatctctcgtcgggactcatttggcgcgtcaggcgctgttgaatcatgaaattgacatggcgcttgtcggcggtgtcaccttgtatttaacgcctgaatcatacatcagcatgtgtgaagccggaatgctgtctcctgacggccaatgtaaagcatttgataacagtgcgaacggatttgtgcctggcgaaggcgcgggggcgctcgttttgaagaggctgaaagatgcggaagccgatcaagaccatatttacggagtcattatcggatctggaattaaccaggacggaaaaaccaacggcattaccgcaccaagcgcgaaaagccaaatggatttagagcgccaagtgtatgaagcgcacaacatccaccctgagagcatcacctatgcggaaatgcacggcaccggcacaaagcagggggatccgattgaattagaagccctgtcttctgtatttaaggaaaaaacggaccgcaaacaattttgcgccatcggttcggttaaatcgaatattgggcatacgtcggcagcggcaggtgtagcaagcgtccagaaggttctgctgtcgatgaagcatcagcagcttgtgccgacccttcatttcagcacgccgaatgaacattttgatttcgatgactcgcctttatttgtcaataccgaactcaaaccttgggaaacaggcggagcaccgcggcgggcctgtgtcagctcattcggctacagcggcacaaacgctcatctcgttatcgaagagtatcagccgaaagaccggggaagccgtaagcctgatgagacaaacatttttgtgctgtcggcgaaaaaaacgcctcaggtaaaaacctatgccgagtctgtgatcgaatttgcaaaagccaatcagaatatcaatctttcagatatggcgtacacgcttcaaaccgggcgtgatgcgatggattaccgtcttgcggttacagccggttcgagagaagaactgatcagccgtttaaccgattttgtaaacggaaatacgggtgaagggatatatgaagcccatatcaaaacggctaaaaaagaaatcaaagcatttgaaacggacgatgacctgaagcagctcgtaaggtcatggttcgataagaaaaaatacgccaaaatagcggaattgtgggccaaagggcttcagattcagatggactgggacagactctatcaaggcgaaaaaccccgcagaatcagtctgccggcttacccatttgcgaaggagccgtactggctgcctgagccgaaagccgtaccgcaggaaacagaagaccggcgcgaggctgtgcttttagaaaagcaatgggttccgtgtgagatcgattcgtccggtgagaaaagcggcaggacggtcatgattctggctgatgcaaggacagcggagctagccgaagagactgcgaagcatttcgctgactcgttcattctcccggtggattccgctgacactgcagctaccgattggaatcacatagatggcgcaatcgatctgacgggatgcggagacgaagacccttcactggaagggctcagcaggctccagactctgattgatacaagccgccgtcaagcgcggccgctgacgctgctatatgtaacaaaaggtttggagccatttcagcagaaaacgattcaacttgcaggagcggcaaaaagcggcctgtacagaatgcttcaaaatgagtatcaaaacgtgacgtccagacacatggatgccgatacagatacggatcagcagacgcttgccaaacagattgcagatgaattttcggccgacagcacagaaccggaaatctgccgccgaaacggacagagataccgtgctttattcacggagattccaagcgggaagcgagcagaacataagcccgcaccgtttcctgaagaccatgtgcttttcattaccggaggcacgaggggactgggtctcttatgcgcccgccatttcacagaaaaacacggggtgaaaaaagtagtgctgacaggcagggagccgattcctccccgtcatgaacggtcagccgttaatacgaggctgaaagagaagatccgggcgattgaagagctcgaagcaaaaggagtgcaagtagcggtgatcagtctgaaattgtctgatcatgatgcggcggcagcggaaattgaaacaatccggcgcacgatgggaccgataggcggcgtcattcactgtgcgggatcggttaattcagtaaatccggcgtttatcagaaagactgccgacgacatacaagaagtgctggaaccgaaagtcgccgggcttcaaacattaagcagccttttcacaaatgatcctttgcaattttttgtcttattttcttcagtatctgcagtcgttccggcgctcgcggcgggacagtcggattatgcgatggcaaatgcgtacatggattatttcgcggcggcttatcagcatcaatatccgctggtcagcattcaatggccgaactggaaggaaaccggattaggtgaagtccgcacagaaagcctgaagcagaccgggctgctgggactgtccaacgcggaaggacttgacttgctggaccgcattttatctgaaaacatccgcccgtccgttctgccggctatcgttgatcggagcgaatggcatcctgacaggctgctctgtcaagagaaaacacaaaaagccgtaataaacggcaaagccgcggaaaaacattcagacagagacctgcttcttgatacgaaggcgtggcttgtgaaactgttttccgatgagctgaaaatcgctccggaagaactggaaacagacgaaccgtttcaggattacggggttgattcgatcattttagcgcagcttttacagcaaatgaatcaagcgctcaaagaggacttagatccgtctgttttatatgaacatccgacgatcgacgcgtttgccgaatggcttgtatccaatgggcagccgctgcttgcagaaagggaggaacctgatacagaaatgttggttccggctgccgtacaaacggttcacactgaacaaaagcgtgatgccgtaactgaagacattgccgtcgtcggaatgagctgccgttttccgggagcggaatcgttagaccaatattgggacttgctgagaagcggcaggtccgcaatcggccccgtcccggcggaacgtttcggatatgcaaatcaatacgttgcgggattgattgacaatatggatcactttgattccgaattctttttcattccggaaaacgacgcgaaagcgatggaccctcaggcgttggcggtgttggaagagagcttgaaactttggtgtcatgcgggctatacccgtgaagaaatcaagggcatagaagccggtgtgtatatcggaggccgcagccagcatcagcctgatcctgaaattcttgcgaatacccgcaatccgatcgttgcgggcgggcagaattatttagcggcaaacgtttcgcaattttttgatctgcgcgggccgagcatcgtgcttgatacggcttgttcatcggctttgacaggaatgaacatggccgttcaggcactgagaagcggtgatatcaaagcggcggtagttggcggcgtcagcctgctgaacacggatgcggcgcaccggatgttccaagagcgcggactgttaaacgaaaaaccggcatttcacgtatttgacaagcgttcaggcggagttgtcctcggtgaaggtgtcggcatggtgctgttaaaaacagtaagccaggcgcaaaaagacggcgacacgatacacgcggtcattaaggccgccgccatgaacaatgacggcagaacggcaggcccgtcagctcctaatatgcaggcgcaaaaagacgtcatgcaaagcgcgcttttcaaaagcggcaaaaagccggaagacatcacttatatcgaagcaaacggatcgggttccgcggtgactgatttgcttgagctgaaggcgattcaatccgtataccgctcaggtcagcatgttccgctcggcatcgggtcaatcaagccgaacatcgggcatccgctttgcgcagaaggcatcgcaagcttcattaaagtcgtattaatgctgaagcataaacaaacggttccgtttctctcaggagacgaaccgatgccgcattttgatataacaaagacagattttcatttccataaaacagccggcgaatgggatgcagccagaccgtcagcagccatcaactgctttgccgacggcggaacgaacgcccacgtcattctggaagcttgggaagaaaaagatacccgaatgaaaagaaagccgctgccggtaccgtcattaagacgtaaggctctggtaaaagagcctgacgcaccggcggagaaacaagagcttcctgcgaaaaaaatgttctggaaagcatttaaatga

SEQ ID NO: 8 - DNA, 10539 bp, Bacillus amyloliquefaciens
atgatatcggaaaaaatcaatatctcactggatcatcccatcatgaagggccataaggtctttggtgaagcggtattgcccggtcttgcttatatcgatttgatttatcaggtttttcaggaacacggttatgtatacaatgagctcgaattgaagcacgttacgatttatcagcctttaaccgtcagtgaaacggctgcaatcattctgaccattcaatgttctgatagcgaagatggaaagtggctcatctccctttcgggacagaaacagcagggtgacgagctgtcagacgatatacaatatgcgacggctgaaatggtgaaaagagacgctgtgacatttgacgaaaccattgatttcagcgatgtgaagcagtcagtcagatctctgaatcttgatgacatgtataaccgctgcctggatcatgacttggttcattccggcatgatgaaatcgaacggacgattgtacgaaacagaaggcggcatgctggctgagctttcttgcgaagaaggaaaggatttctttttattccatccggcccttttggatgcgagcggaatcggggcgagcggtttgctttctgaaacaaaccggcttcatctgcctttatattatgaatcgttcacggccgccgacaggctgcaatcagagtgcacggcgcgtatcgccgcttccggcgtccgtcaggaaaaagaactcacaacgatgacaatcgagtacttcagccgatccggacaaaaggtggcggagcttaaaaacattacgggaaaagcggttcggctcgaagaagacgagaatacagccgaacagaatcaacaggctgcagctgagccggaaagcgcagatgaaaaagacatcattctgtttttaaagaagcttttggctgataagcttggtcagccctgggagacgctggacgttctagccggctactacgagcttgggctggattcatcttctttgcttgagattgtacaagatatcagtaaaaagacgggtgcggacctcgcgcccactcttctctttgaatatacaaatataaaagaactcgcagcctatctgaaaaagaccgtttcatttgaatgtgcggaggaacgtgcagccgaaccatcacggtctgaagctgaagcaccggacgcgccagaacaccataaaaacaccggagacatcgcgattatcggaatggccggccgctatccgaaagcgaaaagtgtggcggagttttgggaaaaccttaaggcaggcacggattgcatcactgaagttccgaagtcccgatgggattggaaaacatatcaaaacgtaacgtccccgtctggaaaaacagtatcaaaatggggcggtttcattgacgatgccgactgcttcgatccgcagtttttcagaatctcaccgcgtgaggcggaaacgatggacccgcaggagcgcctgtttttagaaacatgctgggaaacgattgaagatgcggggtatacgcctgaaacgctgggaaataaaggggagaaacagcatccgatcggcgtatttgcaggggtgatgcataaagattattccttaatcggcgcagaacaattatctgaaactgatccttttccggtttcactgaattatgcgcagatcgccaacagagtgtcctattattgtgacttccacgggccgagcatcgcggtggataccgtttgttcctcatcattgaccgccgttcatctggccattgaaagcatccgccgcggcgaatgtgaagccgcgcttgcgggaggggtgaacttgtcgctgcacccggcgaaatatctcagctacgggagcgtgggcatgcactcaagtgacggccgctgccgcacattcggcgagggcggggacggctacgtttccggagaaggagtcggtgcggtcttgctcaagccgctggaaaaagccgaacaagacggcgaccgcatttacgcggtcattaaaggaagcgccgttaaccacgtcgggaaagtaagcggaattaccgtcccgagtcctgctgcacaggcagaagtcattaaggcctgtctgaaaaaagcaggtatcagcccgcggaccgtcagctatgtggaagcccatggcacgggtacttcgttaggagacccgattgaaattgaaggtctgtcaaaagcgttcagccaagggacgcaggatcagcaattttgttccatcggttcggtcaaatcaaatatcggtcacgcagaatcagcggcggggatcagcgggttaacaaaagcagcgctgcagcttcatcacaaaacgcttgtcaaatcactgcattctgcggaattaaatccttacctgaaatttgagggatctcctttttacgtacagcagcagactgctccatggaagcagccggcagctgaagaaaacggaaaagtcacacattatccgcgccgggccggactcagttccttcggtgcatcaggttcgaacgcccatatcattttagaagaatacattcagcctgaacaaaaagcgcctgcatctgagagcgatgcagcaaaactcatcccgctgtcagcaagaaataaagacaggcttctggcctatgcggaaaaactagcccgatcgctttcagaaaagacggtgctgtctgaactggcttatacacttcagacaggacgtgaagcaatggaggagagagctgtcttccttgtaaatgacacccgcgacttgaaagaaaagctgaatgattttgtaaaaggaaacgaaaacatctccggtttttggcgcggacaagctgagctgccggaactgagcgaagccgctcctgagacggacgatccgaaacggcttgcggcgttatgggcagaagggaaaacagtagattggaataagctgtacggttcggataaaccgcgtaaaacaagcgttccgacttatccgtttgcaaaagaacgctattggattccgcagcagaaaaaacaggagaaaagcctttcaggcacagtgctgcatccgcttttacaagaaaatacgtcagatttttcacagcagcgcttcacgtctctttttacaggagatgaatttttcctatctgaccatgtcgtcaacggacaaaaaatactgccgggcgtcgcctatttggaaatggccgcagaggcggtcaaaaaggccgctgccgtgacggaaaaagaccacagcgtcacactcagacaggtcgtttgggtccggccggtcatgattgaagacaaaccgaaagaaatacatattgccctttacccagaggatgatggtgagattacctatgaaatatacagtgaggcagaaaacggcgaagatccggtcattcacagccagggtgaagcggtcattaccgaaccgggaagcgcccagattgatatcgagtctgtcaaaacttcctgcaatttaggaatcgtgactgcggaagaatgttacggtgcttttaagaagatcggtctggattacggagaggcatacaggggcgtacagcaattgctgatcggagaagaccgattattggcccgtatttcactcccagcagccgtacatgacaaaaaagaacagtatacgcttcacccgagtatgatggacgccgcttttcacgcatccgtcggtctgcttgtccggtcgatacatgacgatgcagacagccacgagctttctttgccgtttgcgatgcaggaagtggaagtgttcggaccgtgccctgaacatatctggtcatatatccggtacacagaaggcagcaaagcagatgacaacgtaagaaaatttgacattgatatgtgcgatgatgccggtacggtttatgtcaggatgaaaggcgcgtccgtccgggcgcttgacacaagccaaacagcgggcggagaaaccacggaggcgcctgtcggccatacgatgctgattccggcttgggagcctgttgcgcttgaaaaagccggggaagcagcggtggataagcagacggcagtattcggcggaacggattcgagacatacgcgcataaaagaacatctgccaagcgcttcctattatccgatcagaggtacggatgatatcacatctcttacgcacatgcttgaaggacaaaaagaactggagcaaatcatttggcttgctccgcaggctgagagtgctgacagcctcaaggaacaggataggaacgttcttcatgtgttcaagctgatgaaagcattacttgccttagggtacggagaaaaacgtttggactggtgcatcgtcacagcaaatgcccagccgttttcggatcaagacagcgccgacccgtcacaagccgcagtcagcggacttgccggaacaatggcgaaagaatatccgcattggcatatccgcatgattgacacagaaacggacggcagctggccgatcgaagaggcgctcagtctttctgccgggaaaaacggtcaaatttgggcgtaccgcaaccagcagctgttagagcggcagctgataccgcttgacaatatgccgaaaaacggcacgctgtaccaaacaggcggggtttatgccgtaatcggaggcgcgggctatctcggagaggcatggagtgaatatatgatccgccgctacaaagcacgcatcatttggatcggaagaagcacactgaatgattccatacaggcgaaactcgaccggctcagcgcattcggcccggcgcctgaatatgtttccgccgatgccgctgacagtgcttccttagagagggcttatcagttcataaaagagcgccacacaaaaatgaacggaatcatccactcggcaatggatctgtctgagcaaagcttaaataatatgaagacagaagagttcaaatcggtgctgcgggcaaaagccgatgtaagtgtgtccatggcggaggtatttggacgggagccgcttgactttgcgctcttcttctcctcgcttgtggcatttattcataacgtgaaacaaagccattatgccgcaggatgcgctttcagtgacgcatttgcgaaagaacttgatcagacatggaattgtccggtcaaagtgatgaattggggctattggggcaacagcgaagcggctgaagacggcgattttattcagctgatggatcagatcggattgggattgatcgaaccggacgctgcaatggaggcgcttgaaacccttatgacgctgccggttaatcaagcagccttcattcatacgaccaaaccggtcgcagtcgaagggatgagtgaaacagaaaaaatttctgtttatccgcctcagccgattgtcttttcacaacagccgaaggtcgctccgcccggcgcacgtcttgtaagagaggctgagcatcaggagctcgatgattttctgtatcagcttttgatcgcccagcttaaagaagcggacttgctgacgccgcaaagcctaaaagtgtatgagcccgagcatgaatactttaaaaaatggctcaagcaaacgatcgccttccttctggaacatggctatctggaagggaatgcggattcatttacactgtcggcacgcgccgaaaacacaaccgccgcgctgtggcaaaaatgggagcgggaaaaggcggaatggctcaataacgccgatacgaaagcgatgactgttttagccgagacgatgcttaaagcactgccgggcattctgcgcgaagacgtgccggcgacagatatcatgttcccgcattcttcaatggaacttgttgaaggaatttataagcgcaaccaagtcgcggattactttaacagcgccctgtcagatacattaacggcatatgtgaaagaaaacatcaacaaacattcaaaagccgagatccgcatactggaaatcggagcgggcacaggcggcacaagcgcaggcatatttgaaaagctcaggccgtatcaaaaccatataaaagaatactgttacaccgatgtatcaaaagcttttctcgtgcatgcggagacggaatacggcaaacggaacccttatctgacgtataagctttttaacgcagaagcgccgcttgccggccaggatatagagccgggcgcatacgacatcgtcgttgcggccaatgtgctccacgcgacgaaagacatccgccagacgctgagaaacgcaaaagccgttttaaaagacaacggtttgctgctcttaaatgaaatggcgggaaacagcctgttcccgcatattactttcggactgcttgaaggctggtggctgcatgaagattctgcccttcgtattccggggtgcccgggcttgtatccggaaagctggaagcgcgtgttagaaagtgaaggctggcattctgtcttcttcccggcagaagcggcacatgaattaagccatcagattatagcggcaaaaagcaacggcattgttcgccaaaaaacgcttcaagcaaaggccggaaaacggaccgctccgccaaaagcatcacatgcgccggaacttcaggggcaaacaacggtgcaaaaaggcgacacgtctttaatagaaaaaagcaccgagtacttgaaaacgttaatcggagaaacattaaaaattccgccggctcaaattgatccgaaagcgccgcttgaaaaatacggaattgattctatcgtcgtcgtacagctgacaaacgcgttacgaaatgtattggaccaagtcagcagcactttattttttgaatatcagacgattgaggcgctcgtcgctcacttaatcaaaacgaaaaaagacgcgatgatcaaactgacgggacaaacgcctgttcaggaagaacggaaggcatcaccagaaccgcaggctgtacagccggctaagccgcttcgcactgactggcggcggagggaaaaacgggcagccaaagagaccgtccggcaaagttcggttcgcgacgtggccatcatcggtgtttcaggacgctacccgcaggcaaaaacagcggctgagctttgggcaaatgttaaaaaaggaaaaaactgcatagaagaaatcccgcgggaacgctggaactggcaggattactacgatgaagagaaaggtaaagaaggctctatttatacgaaatggggcggcttcattgaggatatggacaagtttgatccgctgttctttcagatttctcctttggaagcagagcgaatggacccgcaggagcgattatttttagaaacggcgtatgcgagtatcgaggatgccggctatacgcctgacaacctgtgccaaagcagaaaaatcggtgtttttgccggcgcgatgaacaaaaactacccgacaggctacggctattggtccattgccaacagggtatcttacctttttgatttccaagggccgagcattgccgttgatacagcctgctcctcttctttgacggccattcatctggcgcttgagagcatatacagcggttcaagcgaatgcgcgattgccggtggcgtaaacatagtgaccgacccgatccattatatgaatctgtcagtcatgaacatgctttccgcaggcgacacgtgcaaatcgttcggagaccgcgcggatggttttgtagacggagaaggggtaggcgcggtcgtattaaagccgttagaccgagcgattgccgataacgatcacatttacggtgtaattaaaggaagcaccattaacagcggaggcaaaacgaacggatacaccgttccgaatccgaatgcgcaggccgccttaataaaagaagctttccaaagggcaaacgtcccggcgcgcaccgtcagttatattgaagcccacggtacgggcaccgcattaggagacccgattgaaatcgccggattaacgaaagcatttgaagaggatacaacagacaaacagttttgcgccatcggctccgcaaaatcaaatatcggtcattgtgaaagcgccgcgggcattgcgggtctgacaaaaatattattccagctcaaagaccgccaaattgcgccgtccctgcacgcggaacagctgaatccgaatattgatttcgcgtatacgccgtttgccgttcagcatgagctcggtgagtggaaacgtcccgtcatcggcggcagagagcttccgagaagggcggggttaagcagcttcggcgcgggaggagcaaacgcccacctcgtcatagaagaatatacggcagataaagagcctgatgagacgccggcagatcagcccgccatcatcgtcctgtctgcgaaaaacagcgaacggctcactgaaaaagcaaaacagctcagagacgccatccgtgaaaaaagatacagcggccgcgaccttatgagcataacctttactcttcagactggccgggtgccgatggaagagcgtctcggttttatcgtccattcacttcatgaacttgaggaaaaactgaatggatttatcgccggacaaaacagcgccggactgcaccgtgacggcacggaagaaaacaaacggatgcttgccgtattttcggcggatgatgatatggaaacaatcatagacgcttggatcagcaaacggaaatattctaagctgcttgagctttgggtaaaaggattggacatcgattgggcgaaattatacggagaccgtcctccggcccgcatcagcctgcctgcttatccgtttgcgaaagaacgcttctggcatcagcagcctgtacaaagcaaggccgcgcccgttaagtcgccggctgatgaaaatgatccggcggaacaatttgaagtaatggcttttagagaagttcttaaagaacaggctctgccggctgccggagaaaagcaagtcaaaacggtgatttctttcttatctgatcagaagaagcagcaggaagccatctctgtgatcaaatcgtatgacaatgacattgacgttatttttatcgctcagggagaccggtatgaggctctgtccgccgggcgctgccaagtggtgcgggaagatccgcaatcatatgtgaacgcttttgcggatattcaaaaagagtacggtgaagcttctgccattttgtatttatgggcgcttgaagataaaagctgcatcgaagattattcatgtatcgttcatattctcaaagcaatggctgagaccggtcagcagccggaaaggctgttattggcggggggatatgcatccggtctggaaaggtgctacttggaatcatggatcggctttgagcgttcgctcggcattgttctcccgaaaacaagtgttaccggccacttcagaaaagccggggcggccataacggactggatgagaaccatttgggacgagctgaaagctgaaacagaccaaaccgttttcttggaagagggaaaacgatacgtctatcacaccgaaccaaccggacttacgaaagaaaacagcagaatcagaaccggcggaacatatctcattacaggcggattaggagggctcggctacttatttgcccgccatctcgcccggaactaccaggcaaatgtcattctcacggggcggtcgccgatcaatgaagaaaagaaagaaaaaatgaaagacctggaaaatctcggcgcgggtgttctttacgccgaagccgatgtttgtgatccgatcggaatgggcgactgcatcaagcgcgcgaaagaacgattcggagccattcacggcgtcattcatgcggcgggcatcgaaagcagaacatccgtctttgaaaacgaaatcgggaatttccgcagtacgcttgaaccgaaaataaacggaagcctgcttcttgacgagtggctgaaaaacgaaacgccggatttcatctgctacttttcatccacatcggctgttttaggggatttcggctgctgtgattacgcggtcgcgaacaggttccaaatggcgtatgcccactataaaaacgaacaatcagaaagcggaaacgcttttgtcataaactggccggtctggaaaaacggcggaatggccgtcggcggtgaagagacgacgggcatgtacctgaaatcaagcggccagcgcctgttagaaacagatgaaggcattgagctttttgagcgcatcatttctcaagacaacacccagtgccttgtcattgtcggcgaaagaagccgtgccgaacgatttttaggcatacggcgggtcgattcatcaaaaccggatgcgcttttggaaaccgtgagctgccgcatcccggaagaaagcacggaaaaacggctcgaagcggatttgaaagagcagatacacagtctgctgaaaatatccaaagataaactcgtcctgcataaaaactgggcggaattcggatttgattccatctatttagccaaatttgccgctcttttgacgagtcattacggcattgaagtcactccggcgttattttacagctatgccacgctcggagacgtcatgagctattacctgacagaacataaagaaacgatcgaaacgttttacagaacagaagaaaccgaaacagaagccgccgctccggataacaaggagtatacggatcaggagatcatcgccatgatgaaacaggtctctgaggggacattagattttaaaagagtgcaagacatcatagaagggagtaagacctatgaatcataa

SEQ ID NO: 9 - DNA, 16302 bp, Bacillus amyloliquefaciens
atgaatcataaagaacttctcgatgcctacagatcaggaacgctcaccatcgctgaagtagaacaaaagcttcaggcttctaagcggacaactgcaaaacgtccgctttcagaaggccagaaaggtttgtggatgcttcagaaaatgtttccggagatgagcgcatataacattccgttatgctttcgttttcaaaagctggatatcgagacattcaaaaacgcccttttgtccgtgcagcgccagcaccctattttgaccagtgtgataaaagaggaaaacggccttccgtatcagatagaagatacggatcggcattttccgattgaagaaatggatctttctgacttaagcgggcgtgatgcgcttacgtttattaaagaaaaagcaaaagaaccgttcttgcttgaaaccgggccgctcctgcgtattcaagtattccggcaggcgaagcgggagccgattgtgttaatcaccattcaccatatcatttttgacggcgtctctcttatgacgtttatgtcggcgctgctggacgcttatgcaaaagcaagtgaaggcaaagaacctgtcatgcttcagcaggccacagattatcatgactttgtgaaatgggaaaaagagctgcttgaaagcgaagcgggaaaaacgcatctctcttattggaaaaagcagctggaaggaccgctgccggtgcttgagcttccgtcggattacccgagatcatcggttcaaacttgcagggggcaggcgtatcagcgcttgcttcctgatgaactgaaccggcagattaaagtttgcgcccaaaaaaataacgtcaatgaatccgttgtgtatctcgctattttcaaaggactgctctatcaatatacgaatcaggaagatgtcatcgtcggcatgccgacgatggggcgttctgaggctcaatttgagtctgtcatcggctactttattaatatgatcgcggtcagaagcagggagacgggcgccaaatcttttacgtcgtttttgaaggagctgcagctgacactggcagaagggctggaccatgccgcatatccgtttcccgcgcttgtaaaagaattgaagacggaacgctcgcctgcggtgtcgcctgtttttcaaaccgcgtttttctatcaaaacttcttccggacggaaggagcggaaaagctggcggaactatatcaatcactcggcattgaaatgatagaagacattcatcaggaaggcgaatttgagcttgcgcttgaagtctataaaaaagaaacgggaaccgtcctgcaatttctgtacaacccggatgtctacagcccttctttcatcgaacggctggcggagcattatatgaaactggcggagaaggtgctttctgatccggagatcgcttttgaagcctgttccggcctgctcaatcaaaaacgggatgaaagccgttcatccgatcagccgaatgagcgatgcattcatgaagtgtttgcggaaaaagccggacgccaccctgacagagaagccgtcacattcggtgaaaacagtcttacatatcgcgaattagatgaacgaagcacggcgctcgctgtgtacctgcaagaaaagggtgtcacaccggaaagcccggtcggcatttgtatggagcgttcttttgagatggtcatcggcatcctcgccattttaaaagcgggaggcgcgtatgtgccgctggacccggcttttccaaatgaacggatggcccacgtgttgactgacagcggcgtgtcaatcgtattaacgcaggaaaagacgcgcggcatgattgaacagttggccatcggcggtgtgagcacggtggaaattgaaaacagcggacgttctgtcagcggggcaaaactgacgccgcgttcaaaaccgcatcatctcgcttatattctatatacgtccggaagcacgggaaaaccgaaaggcgtcatggttgagcacagggccatcatgaatacgctgcagtttttggaagctgagtatccggtcgctcaagaggatgcctatctgttaaaaacgaattacgtatttgacgtttcgatttctgaattattcggctggtttatcggaaacggacggctagtcattttgccgcctggtgcagaaaaaaatccgcagctttgcatggaacatatccaaacccaccaggtcacgcatctgaattttgcgcccgccgtgtttaatgtatttctggaaacggtaaagcggcatacggcatttacagaagacggccctgtgaagtatgtgatggtggccggtgaggcgtttccgaaagatttagtcaaaaagtcagtttccatcttcaaacatgccagaattgaaaacatatacggcccgacggaaacatccatttatgccgcttactacagctgcggccaaaaggaaatcacaagcaggaatacgccgatcgggaaaccgattcacaatacgcggctttatatcgtcgatgaaaagctcagccccgtccctgacggtgtggccggggagctttgcgtggccggcgccggtctggccagaggctattacaatcagcctgagctgacggcggcatcatttattgataatccgtttagtgaaggagaaaaactttacaagacaggtgatttggccagatggctgcctgacggaaatatagaatatgccggccggattgacagtcaggtgaaaattcgcggcttccgcatcgaattgggagcaattgaaacgaaattgagcgaacatccggatatccttgaccaggcggttgtggttcaggaaaagaacggacataaaaagctgattgcttattatacggcccgctccggtcatacggcagacgaaaaagcgctccgcaaccatcttttatcaagcctgccggattatatggcgcctgcccattttatccgtctggatgcgctgccgttaacaccgagcggaaaagtgaaccgcaaagacctaaaacagcgcaaggtaatcgttcaccggtctgaaaagacgggcagactgccgttatcagaagcggaagcgaaagtgaccgcaatatgggaagacctgctgaatacggcgggcatcaaaaccgaagacggctttttcgatgtgggaggcgactcgcttctggctgttgcggcggcagagcgcattaagcaggagctggaatgcgattttaccgtcacggatttattcgaacactctaccattcaggcgatcagccgatttattgttgaaacaaaacaggcagctgaatcagaggtgtacggccaagatgagagagaaagagaacaaacaccgcaaaacggggtgccgtcttattttgatgacagtgtggcgatagtggggatttcctgccagttccccggcgcgaaaaatcatcatgagttttggaagcagttaagagaaggaaaggaaagcgtccggttctactcggaagaagaacttcgggaagccggcgtgcctgaagatttgatcgaaaatcccgactatgtgccggcattatcgacgatagaaggaaaagatctctttgaccctgagttctttcacatttctccaaaagacgcagaatttatggatcctcagcttcggcttctgctgcttcattcttggaaagccgtcgaggacgcgggatatgtatcgaaagagattccgaagacaagcgtatatatgtcggcaagcaataattcgtatcggtcgcttttgccggaaaaaacgacagaaggccatgaatctcccgatggctatgtgtcatgggtgttagcgcagagcggcacgattccgacgatggtttcccataagctcggattaaaagggccaagctatttcgttcattccaattgctcatcatcattagtcggtctttattcagcctacaaaagcataacgagcggagaatcggaatacgcgctcgtcggcggagcaactctgcatgcagcgaccggcattggctatgtgcaccaaaacggattgaatttttcaagcgacgggcatgtcaaggcatttgatgcttcagccgacggtatggcgggcggagaaggagcggccgtcattctgctgaaaaaggcgtcacaagccgttcaggacggagatcacatatacgccatgctcagaggcatcggtctgaacaatgacggagcggataaagtcggtttttacgcaccgagtgtaaaaggtcagactgacgtcatccagcatgttttagacagcacgaatattcacccggagacgatttcctatatcgaagcgcacggcacaggaacgacattgggtgatccgattgaaatgtccgcgctgcagcaagtgtataaacgttacacggaccgggagcagtattgcgggatcggctccgtaaaaacgaatatcggacatttggatacagcagcaggtctggccgggtgcatcaaagtcgccatgagtttatatcatcgggagctggcgcctaccattaattacacgtcaccgaatccgaatatcaaattcagcggttcgccgttttatgtggcggataagagaaaaccgcttccggagagagagacgccgcaccgcgcagcgttaagctcattcggactcggaggaacaaacgcacacgccatttttgaacagtatgaaaacaaaacgatcagcggatcaaaggacggacagccgccttatatcgttccgctgtcagcgagaaataagcagcgtcttactgcctatgcatcctgtttatccagctttctggatgaaaccgaaaacgatgtttccttgcatgatctggcatttacgtatcagaccggacgtgaagcgatggaagagagagccgtttttatcagtcatgatcgtcacgatttgaagcgtcagctgcaggatttcatcaatggcaatgaccaaaacatcttacggggcgaaaaagtgagaagcagagaagtgtcagctcaggagatggaagagcttgcggcatgtcagacacgggatgaaaagctgaaagcactggcggcattctgggttgaaggagcgcgtgtcgattggggcttattgtatcaggattccgcaccgcaaaggatcagtgcgccgacctacccgttcgcagaggagcgcttctggccggaagaaacggtgaagcaaacggataccaagcttttgcatccgctggtacatcagaatacatccgtcttgcaggagcagcggttcagttcggaattcacgggaaaagagtattttattgccgagcatatcattaaaggaatggcgatcgtgccggctgccgttacccttgagatggcacgggccgcggcggagaaaagcatcggcggggcaccggaggattctctgggcttgcgcttaaagaatattgtatgggtccgtccggtgatagccgatcaggaggctgtcagtatccatatcggactttatgaagaagaggacggacagattcaatacagaatgtacgggggtaacgattctatcggtgaagaagctcctctttataatcagggcatcgctgaaatcatccggaaggaaccggaggaagcggcggatattgagcagctgaagcgccggtgcacacagggaacaatcgaatctcaagcgttttacaaaggaatgatcggtgctgattacgggcccggatacaaaggggtgcagcttgtatacaaaggcgaagaccgtctgcttgctaaattgaagcttcctgaatccgtctctcatacaaaagctgattatatccttcatccgagtatgatggacggtgcgcttcaggccgccgaatatttgcaaaatgtgacgagagcgctgcttgcagaaagcggggaaccattcaaagccgcattgccgtttgctttagaggagctggacgttctgaatccgtgcgctgatgatatgtgggtgcacgtcacattcagtgcgaacaataaagccggcgatcctattcaaaaagccgatattgatctatatgacaataacggcgggctttgtgtgcggatgagaggtttctcaacgcggatcatggaagaaaacgcgggtgccggcgtttcggtcagccgtacggacacattgctcgctgaaccggtctggaaagagcagccggaaccggccgatcacgagccaacgtatgaagagcatatcattttcctttgtgagtacgatgaagccgtaaaacaagctgtcgaagcaaagcttgaacatgcaagcgtcattatcttggacgatcgtcccgcatctattgcagagcggtatcattcttatgcgggtcagctgtttgaccatatcaaagccataatgaaaagcaagccgaagggccgcgttctttttcaggctgttacgcagtcagcaggcttacagcaggtgttctcaggcattaccggcctcttcaaaaccgcatgccttgaacattccaagctgatctgtcaaatgatcgaagccgaagagaaagaaagtgctgaaggcatagcagaaaaagcgttcgaaaaccgtatgcatgccgaaagcgtccatgtgaaatatgaaaacggcaagcgctttgccgctgattgggaagagatggatatttctcaagcggagccggacattccgtggaaagacgaaggcgtttatctcattacaggaggtgccggaggtttaggtctgatctttgcaaaagagatcgcaaaccgtacaaaagacgccgtcatcttactaaccggccggtccgctctcagagaaaagcagaaagacatgctggaaacagtacgtgctgccggcgcttctgttttctacgagcagaccgatgtcacggatgaagcagaagtataccgattgatccgcaatatacgcaaacggcacggcaagctggacgggattgttcacagtgccggcatcataaaagacaactacatggtgaagaagacggaagaggaataccaacacgttctcgctccgaaagtgaagggccttgtgtatcttgatgaagcgtcacaacatgagaaattagatgtctttattgtcttttcatccttatcaggtgtgcttggaagcgtcggccaggctgattatgcgtctgcaaatgtctttatggagatgtatgccgagtaccgccgttctttacaagccgcaggacaaagatacggaaagacgctttctgtcagctggccgttatggaaagaaggcggaatgcaggcaggaaagcagacggaaaatatgctgatgcaggaggcgggaatcgcaccgctcggcacagatgccggcatcaaggctttatatcaaggcttgatgagcgggacagcccggatgacggttcttgaaggagatgtgaaacacatgaaagcaaaactgttccgcaaagagaatgatcatccggaagcagagaccggcgtacgtcatgacattgatgctgacagcctgcttgaaaaagtgaagcatctgttaaaacagcagacggcctcactgttaaaagtaaacattgacaaaattgatcctcatgaagaaatgacaaaatacgggctggattctatctcgatgactgagtttacgaaccagttaaacaaaacgtaccggcttacgttaacgccgacgattttctttgatcatccgacaatccatgaatttgccgttcatttaatcagtgaatacgaagaggaatttgccggacggtttgcggtaaatacaaaaacggttcaatcggcaagaccggtaaaagagactcaaatcacgcgcccggccgttaagcgacgccgcactctgcctgagacacttccgcagacagtgcagcgggaccaagggccggaaccgatcgcaatcgtaggcatcagcgggattttcccgatggcagaagacattgaatctttctggaagaacctgaaagaaggcaaagactgcatgaccgagattccgaaagaccgctgggactggcgggactattacggggatcccgcaaaagaagccaataaaacaaacgtcaaccaaggcgggtttattgacggaattgctgaatttgatccgttatttttcggaatttcaccccgggaagcggagcaaatggaccctcagcagcgcctcttgctgacgcacgcgtggaaagcgattgaagatgccggatatgcgtcaaagagtctctcaggcacaaaaacaggtgtgtttatcgggaccggaaataccggctacggttctcttttagccaatgccgatgcggagattgaagggtcatccgcggcgaatacaagtccgtctgtcggaccgaaccgtgtcagctatacgctcaatctgcacgggccgagcgagccgattgataccgcctgctcaagctcgctcgttgccatacaccatgccgtttcttcaattgaagaagggacgtgcgacatggctttggcgggaggaatcaatacgattgttcttccggatgtgtatatcagttttgacaaagcgggcgcgctgagtaaagaaggaagatgcaaaacgttctctgatcaggctgacggttttgcccatggtgaaggagcagggctctttttcctcaaaaagctgaaagccgctgaggcggatggggaccatatttacggcgtcattaaaggaagcgcggtgaatcacggcggccgcgcggcatctttaacaacgcctaacccgaaacaacaggcggaagtcattaaagccgcatacaaaaaagcgggcattgatccgaaaacagtctcttatatcgaagcccacggcaccggaacagaactgggagatccggttgaaattaacggcctgaaacaagcgttcagctcgtttgcagacggaaaagagaaagcggccggctattgcggcttaggctcggtcaaaacgaatatcggccatttatcccttgccgcgggagcagcgggcattattaaaattttgctgcagatgaagcatcggacattagtaaaaagtctccactgtgagacggtaaatccgtacattcagcttgaagacagcccgttttatctggtgcgggagacgatggaatggaagacgcagacggacgaacgcggaaacgaacagccgcgccgcgcgggcatcagctcattcggcatcggaggcgtaaatgcccacgtcgtcatcgaagaatacatcccagaagacagagaggaagcagatgactctcatatcgacccatgtcttttcgttctgtccgccaaaaatgaaaaacgcctgaaagagcaggcaaaacagctgaaacgtgcgcttacagaaaagcggtacgatcaatgcagcctcgtttctatggcgtataccctccaaatcggaagggacgccatggaagaacgcctggcggttattgccggatccgcagaagaactgcaggaaaaactgtcacagtttatcactggagataagcacgcggatttgtacagaggaagaatagacaaaggcaccctgcaaatgctgacggaagatgaagaaattcaagaagccgttgaaaaatggatggcacgcggcaaatacgcgaagctgcttgaattatgggtcaaagggctgaacgtcgattgggcgaagctgtatggagaccggccgcctaaacgtatgagcctgccgtcgtatccttttgcaaaagaccgctattgggcggcaaaaagcaccggaaaacacgaaaaaacaagcactgtgtctgccggaaaacaagacgcgcttcatccgcttctgcacagaaacacctcaaaccttgcagaacagcggttcagctcaatctatacaggagaagaattttaccttgcggaccatgttgtaaaaggcgtgccgattttaccgggggtggcccatcttgaaatggcgcgcgcagcaatggaacaggcatctgaagttccggccggacaaggaagcgtcaagcttaagaatacggtttgggtgcggccgatcaccattgaagacgagccggttcatgtcaacatccgtctcattcctgaaacggcaaatgacgttcaattcgacatttacagcggtcaggaaacagatcagccgtccgtttacagccaaggggcagtatcagtccgccctgacgccgaggctccggtcattgatctccaaaagctgagatcacaatccgccgagacaccgttttctgcagatgaagtatacgatatgtaccgcaaaatcggttttgattacggcccggctttccgtggtgtcaaagcaatttatacaggtgatgatttcgtattgggcagactgtcacttgatgcggcttgtgcggacacggtacaggagtatgtcatgcatccgggcttaatggactctgcattgcaggcatcaagcatattaactggaacaaatgatgaaaaactgatgcttccgtttgcggttcaggagcttgaagtattcagcgcctgcgctgccgagatgtgggtttacgccagactttctgaagaccacaaagccggccagattgaaaaacgcgatcttgatatttgtgatgaaaacggcaaggtgtgcgtaaggctgaaaggtttgtcattcagaacggctgaaaaagaacctgagaaaaaagaaaaagctccgggaacgctcatgcttgagccggtatggacccgaagtgatgaaactgctgttcaaacggaaccggtttttgcccggcatatcgtaatactttgtgacacagacgccggattaaaacaaaccgttgaagctacgggagctgaatgtattaaaatgacctcccagcacgggaaagcggcggcgcgcttccgtgattatacaggacggatattggagacgctgcaaaaactgctgaaggaagagacggatggagacgtcctcattcagacggttctctctaaaacagataagcatcagctgctctcaggcatttcagctctgttaaaaacagccggcctggaacataaaaaactgaaaacccagctgattgaaatagaaccggcggaaaacggcagaatcacagacatcatcagagaaaataaaacacgggctgatgacagccatatcaaatatgaaaacggcatccgatacagcgcagatgtcaaagaaacggccgtgtccggcggcaaaataagcattccgtggaacgacggaggcatttatctcattaccggaggagccgggggcttaggctttattttcgcgaaagaaatcgcccgcaaagtcaaagagccgacactggttttgacaggccgttccgctttaagcgagaatcagcggatgcagctgcaatcgcttgaatcactcggagcaaaagcggaatacaaacaggctgacgtaacaaacgctcaaactgcggcagacctggtcaaaacaattgaaacggaatatggcggccttcacggcatcattcatagtgcgggtgtgatgaaagatcagtatatcgcaaagaaaacaaccgaagagttttacagtgtactggcccctaaaacggacggatttgtcaaccttgatgaggcaaccgagcatttagcactggactttttcatcgttttctcatccatttccggcgtcaccgggaatgcgggacaggcggattatgcggcggcaaacgcatttatggacagctatgcggcataccgcaacgcactggttacggcaatgtaccgccacggtcacacattgtcaatcaattggccgctttggaaagacggcggaatgcaagtcgatgaagaaatggcgaagctcaccgaaaaaagaaccggcgtaacgccgatgcgcactgaaaccggaatagaagctctttacaaagcatggagcacggggaaaacaagcgtgctcgtcatggaaggaaaccgcgccgtaatgcgggaaaaactgcttcaaaagactcccgcagccgcttttgcaccggttaagaaaacggaggaaacgcctgcgccgaaagacctgacggcaaaacttcaggaattgctggcaaaagaggtttccgggcttctgaaaatcaacattgaagaaatcgatatcgaattggagttcaatcaatacggatttgattccattacgctgacggaattcgccaacaagctgaatgacacttatcagcttgacctgacaccgaccgtattttttgaatatgcgacgattcaagccttggcgcatcatttagcagaagactatcaaacacaatttgccggcagcacacagccgaaagaagaaaaacaaagcaacgtaactgatgatggcggcatcagactggcagggaacaaacgatttgtaaaaactgccgtacagcctgttcagactgcggctgaacataaaccggaaccgatcgccattgtgggcatgagcggggtgtttccgaaggccgcaaatattgatgagtattggcgcaacttagaagaaggaaaagactgtataacagaggtgccggcggatcgctgggactggcgggagtattacggtgacccgcttaatgaagccaataaaacaaatgtaaagtggggcgggtttattgacggagttgcggattttgatccgctgttcttcggaatttcaccgctggaagccgaacagatggatccccagcagcgcttgttaatgatgtatgcgtggaaagcgatagaagatgcgggatattccgctaaaagcctttccggcacgaaaacaggcctgtatatcggcacggggaataccggatacggctcactgttttccgaccttgatatcggcggtgcgtcagccgcaaatatgagtccgtcagcggggccgaaccgcgtcagctatatgctcaatctgcacgggccgagtgaaccgatcgacacggcttgttccagctcgctggtcgcgattcatcacgccgtatgcgccattgaaaacgggaactgcgatatggcgatcgccggcggcgtgaataccgtcgtcactccgcaaggccacatcgcttatgacaaagcgggggcgctgagcaaagaagggaaatgtaaaacattctccgacagggcggacggatttgccgtcagtgaaggtgccggaattctatttttgaaaaagctgtcagaagcggaaaaagcgggcgatcatatttacggcgtcattaaaggcagcgcggtcaaccacggcggacgcgcgaactccttgacgacaccgaacccgaaagcccaggcggaagtcgtcagaacggcttatgacaaagcaggcattgatccgagaaccgtcacttatatagaagcgcacggaaccggaacggagcttggcgatccggtcgaaatcaacggcctgaagtccgcctttcaaaagctgtacgaaaaaacaggcgattccgccgtttacgcgagtcactgcggtttgggatcggctaaaaccaatatcggccacctgtcactggcagccggtgtcgcgggtgtgatcaaagtgctgatgcagatgaagcataaaacacttgcaaaaagcctccacagcgaggtcatcaacccgtatatcaagctgaaagacagtccgttttacatcgtacaggaaaaaagagaatggacggcattgaaagacgaaaacggcaatcatctgccgcgccgtgcgggaatcagttctttcggcatcggcggcgtaaacgcccatgtcgtcattgaagaatatatcccgaaggcagaaagaaaaacagctgttttcacacaccctcaaatctttgtgctgtcggccaaagaagaagagcgtctgcaggaacaggcgcagcagctggcgcaagcttcttttacggatgcggatcttgccgatgttgcttacacgcttcagcagggacgtgatgcgatggaagaaagagcggccattatcgcttcctcggccgaagaacttcgggacaagctaaagagctgtgcgaaaggacgttatgatatagcgggcatttataaaggaaaagcgggtaaaacctccctgaaatccattctggcaggcgcaaacggcgaagcggatatcgaagagctgataagccgccgagagaatgacaagcttgcggaattatgggtgcagggcgcggatatcaaatgggacggcttatattctgatatcaagccccaacggatgagtcttcctgcttacccgtttgcgaaagagcggtactggatcacggacaaccggccgaaagcagagaaaccgcaagctgtgctgacgtcgcgggtactgcacccgctgcttcacgaaaatgtatcagatttaacagagcagcggtaccgatccgaatttacgggacaagagacgattttcaccgatcatctcattaaaggcaagccggtgatgccgggcgttgcctacttggaaatggctagggcggcggttgctcaggcagtcaaagggcaaaaggaacgtccgtctgtcacccgcctgaaaaacatcgtttgggttcggccgatcgaagcgggaacccgcccgattgaagcgcaaatcagcctgattccgagggaaaacggtgaaatcgtgtatgacatttacacggactcacaaacagaagcaagcggcaggcagatacactgtcagggggctgccggcattctcagcgccggggaggctccgcaccgtgatataggcgcactgaaagctcaatgcggcctcagtatactgacttctgaacagtgttacggcttattcagcaaaatcggcattgactacggtccaggacaacaaggaattcagcagattcatatcggaagcaatcaagcattagctgagctccggctgccttccttcctcgaagaaacggcagatcaatacgttcttcacccgtgtatgcttgattcagctttgcaagcgacaatcggactgaagctgcacgcagatgataaccagctgtcactgccgttcgcccttgaagaactggacattttcagcccgtgcacgacaagcatgtgggcgtatgtcagctcccggggaacggatgaaaaaatacagcgtatggacattgacttatgcgatgagacaggacgggtctgtgtcagaattaaagggatttcttccagaatgatggaaggccccgcgccggaaacatcattaaatggagcaaaccttctggctccggtgtgggatctcgcggaattccgtgatcaaagcccggcatataagacggaagaggttgtctttatcgcgggggatgaggaaagcgcggcttttcaattgcatcatccttctgcgaaacggcttcatatcaacccggaggatgaaatagaggctgttgcggctgaactgaaagcaacagggtctttcgggcatatcgtatggatcgctccttttcacagcgctgatgacatggtgaaagctcaagatgcgggcgtcatccagatgtacagagtgatcaaggcaatgcttgctttaggatatggaggcaagcgaatcagcttgactgcagtcacaataaaaacacagtccgtacagccggcggacattattgatccggctcatgcaggcattcacggtttggtcggctcgctcgcaaaagagtatccgaactggcaaacaaggctgattgatgccggatgtccggaagatgtttcaaaggcggagctgttctcagcgccgtttgatgagcaaggcaatacatgggcgtaccggaacaatcactggtacaaacagcgccttattcctgtacagcagccggtgtcaagccggcccgcttaccaaaaaggcggcgtttatgtcgtcatcggcggcgcggggggcatcggcgaggcatggagcgaatatgccgtgaaaacatatcaggcgcgagtcgtttggatcggcagacgaaaacataacagcgccatccaggaaaaaatcgacagactcagccaattcggcccggcgccgatttatattgaagctgacgcggcaaatccgaatgagctcacacgggcgtatgaaacgatcaaacgcacgcacccgcaaattaacggaatcatccattccgcaattgttttagaggatcagagcctggcaaatatgccggaaagccgttttaaaagcgtgctcgctgcaaaagtcaatgtcagcgtgaatatggcccgcgtgttccgaaatgagccgctggactttgctttgttcttctcatctgttcaatcatttgcacgagcggccggacaaagcaattatgctgccggatgcagtttcaaagatgcctttgccgctcatttggctaaaacgcggccgtattcggcggcagtgatgaattggagctactggggcagtgtcggaatcgtatcatctgctgactatcaagaacggatgaaacaggcgggagtcggatcaattgaagcgccggaagcaatggccgcgcttgaacgattgctcggcggcccgttgaatcagttcgtcatgatgaaacaggcggaagagccggaggaaaaaccggaagaaatgatagaagtataccctgaacatcacggctcggcgatgcaaaaactgcgaagctatcagcctaaagtaacaagacatgtacaacaattgagttaa

SEQ ID NO: 10 - DNA, 7458 bp, Bacillus amyloliquefaciens
ttgcaaaacactgaagatcttctttacaaactattggcaggccagctgaaatctgtcggattttttacgaaaaacgaaacgcagcctgtcatgcaggggttttacggcaggtggctggaagaaagcatctccatcttggtgcaaaaaggctttttgaaacagtcaggcagtacgtatgtgttaacgggtccgatagagcgtgcggacgtgctgtggcaagaatgggatgaaaagaaagctcctttactcaaggacagcagcaaagaggcgatgatcaccttagtggagaccgctctcagagctttgccggatattttatccggcagagtttctgccactgatgttctgtttcctgattcctcaatggaattggttgaaggaatctataaaaacaatgaaacggcagactactttaatgaggtgctcgctgacacattgacggctttcattgaagaacgcctgaaagaagacccgaacgcggaaatccggatattagagatcggagcggggacgggcgggacgagcgcggccgttttcaaaagattaaaagagtacaaacatcatgtgaaagaatattgctacaccgatctgtcaaaggcctttttcatgcatgctgaaaaagaatacggtcccgacaatccgtatttgacttataagcggtttaatgtggaagagcccccggctttgcagggtatagaggcgggcgcatatgacgcggtaattgcggcaaatgtgcttcacgcgacaaaaaatattcggcggacacttcgcaatgccaaagcggtgcttaaaaagaatgggattttgctattaaatgagatcagcagccattcggtttattctcacctgacattcggccttttggaaggctggtggctgtatgaagacgccaaactgcgcattcccggatgcccggggctgtatcctgaagcgtggcagtctgcgctggaaagcgagggatatcggtacgttacatttatggcggaatcgtcacatcacctcggccagcagatcgtggcggcagaaagcgacggcgttatcaggcaaaagcagccggacgcgcaggcggttgaagaaactgcggcttccaaagagccggctgatttcacagaaccgtctttctcagtcacagaccggactgtgcaattcataaaagaaacgttagccaagtccataaagcttgctcccgagcgcattcagccggaaaccagctttgaaaaatacggcatcgactccattttgcaggtgaattttatccgcgaactggaaacattgacgggcgagctgccgaaaacggtcttatttgaacacacgaatacgaaagaacttgccgagtatttgctggaaaattacccggataagctgcaacaggcattcggagccgaacaagcgcctcctgaaacggagcatccggaacctgcaatgaatgaaccggtgccgcttgtcacggaaacgcggcgatttatcactgaacaaaaagagccaaaagcgccaaagccggaaacagaaaccgtcaccgttaatcgttctgatcgtggcgacattgccattatcggcatcagcggccgctatcccgagtctgaaacgctcgatgagctgtgggaacatttaaaagccggagacagctgtattacggaagccccggaaaacagatggaagtccggtcttctgaaaacaatggcaaaagaaacacgaaaagaagaacgcaaaacacggtacggcggatttctgcagcatattgacgcttttgaccatcacttgtttgacatgcgggaagaccatgtgatggagatgacgcctgagctccgtctaagcttagaaaccgtatgggaaacatttgaaaacggcggttactcgttagaaagagtaaacgaatggcaggaatcagacagcggcatcggtgtatttatgggcagtatgtacaatcaatatttctggaatatcccgtcattggaaaaagccgcgctcagctctaacggaggggattggcatatcgccaaccgcatttcccactttttcaatttaacgggtccgagcatgggcgttacgaccgcatgctccagctcactgagcgccattcatctcgcctgtgaaagcctgaagcttaacagctgttccatggcgattgccggaggggtcaatctcacgcttgaaccgtctaaatatgatgcgctcgaacgggcgaatttgcttgaacagggaagtgagagcaaaagcttcggaaccggcacgggcctgatgccgggagaaggagtcggggcggtgctcttgaagccgctctccaaagcgcttgccgacaaagaccatatttacggggtcattaaaagcagcgcgttgtgccatagcggcggaaggcagatgtacacggctccggatccgaaacagcaggcgaagctcatggcggcttccatcgacaaggccggaatcaatccggaaacaatcagttatgttgaatcagcggcaaacggctctgtattgggagatccgattgaggtcattgccctgacaaacgcgttcgcgcaatatactgataaaaaacgattttgcgcgcttggatccgtcaagtcaaatctcggccatttagaggcggcttccggcatgtcccagctcgcgaaagtgctgctgcaaatggagcgggaaacattagtcccgacgattaacgcaaaaccgcaaaatccgaatatcaaccttgaacaaacggctttttacctgcaggaaaaaaccgagtattgggaacgtctgaaggatgccgagacgggagacatcataccgcggagaagcatgatcaattccttcggagcaggcggggcatacgcaaatctcatcgtagaagaatatatcagcccgaattccgatcaggcccgtcacgacgatgaggaatatattttccctgtgtcagcgaaaacaaaatggagttttgaagcctatcttgagagactggcggcctttttagaaaagacgccatctcttcatccggcagccatcgcatccgcgctgcaaaaaagaacgcatcattcggagtatcgggcggcatttacggctgcttccgttcaggaactgctggaaaagctggccgcttaccgcatggatcagcggaatctcaatttctccgattctgatattggccggcgtgcgcggcaatgggaaaaaggagagagtatcgcgtttgatcagttgcagcaagagcacgccgttcatctgccggtctacgcttttgaccacgggacttcttttcaattcggtgaatcagaaaacgctgccggcgttcaggcggtatacagtgatgacgacccatacgtgaaagggcatcaattcagcggagaaccagtactcgtcggcgctacatataacagtctggcagtcgatcatttttgtgatctttttcctgaagcggaaggaggacaaattaaaaaactaaattacgcgagccccattaccgtaaaaaaaggcgatcgcatcgagctgaaaacagaatcgaaacaaaacggggatggatttgaactgagcataatgcaccgcagccgcacgtctcctgattggagcatagcggcgtatgccgagtgcagcccgtatccgtttcaaaagcagcacgcggatctggcggccattcagcgctcttaccgggaagtgggccggatagatgagttatacggagacggaacgggaactgtcgtgtggggagaagaatttaaaacgatcacccgcctgtacaaaggctctgatgccgttttagcgaaagtgaacctgaacaatagcgactcacaacaagcgcatcattatgccgtaaatccattaatcgccaacactgcttacttggcctcgctttctttattggaccaaacggaggcggaaagctatctgccgttcggcattaattccgtcaccttcagcagcccggaagatcttaccgattgctggctgcacatcaagctggccaaacaaacgggggaaatgattctgattaacgcagatgtcatcagcaggcgatcggaagtggtgatgcgttttgaaggctatgccctgaaacggttccgcccgaaaaccgaacagataaatcagccggcccgaacggctgtgaagaatgcgggagcaccgatttctgccggcagtctgacagatgatatccgcagctacgtccttggcaagctcgccaagatgatggatgaacccgcgccatctcttcgttctgacgcaaatatcatggaattgggacttgattcagcgagcctgatcgggctgacaaaagagatcggccaagaggtggacatcgatgtgaatccaacattgtttttcgagtatccgaccgcggaggagctgacccgccacttcgcgtcagcatatgagagcaagttttctcaattgcttgccgatcataagcattcagaagaggccagggtcacggaaacagaagagcgcactttaccgaagaaacagcatacgccttgtgatcacgccgatatcaagcaaggaatcgcgattatcggtatgtcagctcagtttccgcaatccccggatatccagtctttttgggagcacatcataaacggggatcactgtattaccgaaattccggcggaccgctgggactggcgccgttacgctggtgatgagaatgacacgagtctcagatggggcggttttattgacggtgtcggcgaatttgacccgctctttttcggaatttcccctaaagaggcatcacaaatggggccggagcagtttctgcttttgatgcatacatggaaggcaatggaggatgcgggcctgacaaataaggcattatcaagcagaccgacaggcgttttcgttgccgcaggcaacagcgatccgaacaatggaacagccatcccgtccatcattccgaacaggatatcatatgccttgaatttacaaggaccgagtgaatattacgaggctgcctgcacgtcaaccctagtggcattgcacagggcggttcaatcaatccgtcataacgaatgtgagcaggctgtcgtgggagccgccaatattctgcaatcgccgaagggctttatcggttttgactcaatgggatatttaagcaaaaacggccgggcgaaatcgttccaaaaagatgccgatggctttgtcagaagcgaaggggcgggcgtgatcatcatcaaaccgcttgaggcggcaattgatgacggcgatcatattcacatggtcattaaaggaacaggagtgtctcacggcggtaaaggcatgtcgcttcacgcaccgaatccggcgggaatgaaggccgcgatgaaaaaagcgtacgaagacaccgatgttgatccgcaaaccgtcacttatattgaagcgcacggcattgcctccgaaatggcagatgcgctggagtttaatgcaatcaaagcgggttacggcgagtctgcaaatcaagaggaaagcgcgccttgttatatcagcaccgtgaaaccttgcatcgggcacggcgaactcgcttccggactggccgcccttataaaagtggcgatggccatgaagcatcataccatcccgggcattcccggatttacagctgcaaatgagcaaatggccattcagaaaagccgcttccgtttcaccgaagacaatcaggaatggacacagctcaccgaccatacgggcagactgatcccgcgccgggccgcaattaacagctatggcttcggcggcatgaatgcgcatgtcgtcttggaacagtacagcccgagccataaagacaaagaatctgtgcatgaaccgcagcttatcgtcctttctgctaagagcgaagggcgcctaaaggcggccgcaggtcagctggccgattttgtgcaaactggagatactccgtcattgcgggatattgcttatacgcttcagaccggccgtgaggcgatgaactgccgcctggcactcgttgcccgcaatcaagatgaattgctggaaaaactgaaagagtacagagcattttccgaagaaagcaatggaatgaaaagcttctttacaggcagtcagcagactggatcaggagatatcggcgttttactggaagggactctcggtacagtcgtcacggagacgctgttggctgaaaacaatctggaaaagcttgcattttgctgggtgaaaggtgcggatataccgtgggacagactccaccaaggcaaatctgttcaaagagtgccgcttccgacatatccttttgaaaaccataagtattggaacggcggtcagccagaagacatcgtccttgcaccggtttcacagaatcctcagatccgctgcgagagcgcagctgacattgtggcagacattttggggatggagccagcggaaatggatcaaaataagccgctgactgaatacggtttcgattcaatctcctctattcagctgctgaaaaaacttgagggcggagacagccgcatcagtctggaggctcttcagaaatgcagcactgttcaggagattagcatgctgttaaaaacgaaccaatcgcaggaggcgggccggaattttcccgaactcgtcaagctgaacgatagagaaaaaggccgtcctgttttttggtttcacggcggaacgggaggcgttgaagcataccagccattcgccaaaaagagtcagcgacctttttacggcatccaggcaagaggtttgacagggaaagagccgctgcaaggaatcgaagagacggccgcttcttataagagaatcatccaagcggtacagccgaaaggcccttatgacctcggcggatattccttgggcggaatgtttgcatacgaaaccgcgcgcctcttgcaggaggaaggtcatacggtaaaaagcgccgtcatgatcgacaccccgtacagtgataaatggaaagaaagaaaaccatctgttaagtcagtcatgctgcaaacgatcaatacgatgcttacagcatatcttaagcctgaaaaccttgaagaggcattaatcagcagagaagacattaccgtccaggctgaggaagaagaggtattgaaagaattgatcggccttgccaaaaagcgcggtctgccaaaatccgaagaagcgatccggatgcagctggagcaaatgatgaagacgcagctcgcttacggaatggaagagtatgacatgaagccgctgcctcatccggaagcgctgcaatgctactacttccgtaataaaagcggtttgttcctcggagatcttgaacgttatctgacagcagaggaagaacaaattccgttagaccatgatgcgtattggaaagaatgggaacgccagataaaacagtttcacctgttggacgttcactcttccaatcatgtcacaatgctgtcggaaccaaagccgcaaaacgccattaaagcattctgcgaaaaactgtacgcaaacaaaggaacgataagcgcaaatttccttaaatctttcagaaaaaagctggacgaaacaaatgagttggtaaaacggtaa

(19) United States
(12) Patent Application Publication
(10) Pub. No.: US 2022/0168363 A1
(43) Pub. Date: Jun. 2, 2022

(54) COMPOSITIONS CONTAINING BACILLAENE PRODUCING BACTERIA OR PREPARATIONS THEREOF

(71) Applicant: Evonik Operations GmbH, Essen (DE)

(72) Inventors: Lorena STANNEK-GÖBEL, Bielefeld (DE); Stefan Pelzer, Gütersloh (DE); Thomas Berngruber, Hamm (DE); Claudia Borgmeier, Bensheim (DE); Stella Molck, Bielefeld (DE)

(73) Assignee: Evonik Operations GmbH, Essen (DE)

(21) Appl. No.: 17/429,905

(22) PCT Filed: Feb. 10, 2020
(86) PCT No.: PCT/EP2020/053235

§ 371 Date: Aug. 10, 2021

(30) Foreign Application Priority Data
Feb. 11, 2019 (EP) 19156407.9

(51) Int. Cl. A61K 35/742; A23K 10/18; C12N 1/20; A23K 50/30; A23K 50/75; A23K 50/80; A23L 33/135

(52) U.S. Cl. CPC A61K 35/742; A23K 10/18; C12N 1/20; A23L 33/135; A23K 50/75; A23K 50/80; A23K 50/30

(57) ABSTRACT

Bacillaene producing bacteria and compositions containing bacillaene producing bacteria can be used as probiotic feed ingredients. Bacillaene or derivatives thereof can be used for treating bacterial diseases.

The invention concerns the use of bacillaene producing bacteria as probiotic feed ingredients and compositions containing bacillaene producing bacteria as well as the use of bacillaene or derivatives thereof for treating bacterial diseases.

The use of certain bacterial strains as probiotic ingredients in the feed industry has been disclosed before in the state of the art. The function of probiotics (also called “direct-fed microbials” or “DFM”) is to influence the gut microflora in a positive way by supporting the growth of beneficial bacteria and/or by suppressing the growth of pathogenic bacteria. Ideally, by using probiotics the use of antibiotic growth promotors (AGPs) becomes redundant. But besides that, it is desirable that the probiotic fulfills further functions like helping in the digestion of specific feed ingredients.

Thus in view of the state of the art, there is a need for probiotics which influence the gut microflora in a positive way and beyond that desirably fulfill at least one further function.

Bacillaene is a secondary metabolite belonging to the class of polyenes, which was discovered and isolated from fermentation broths of a strain of Bacillus subtilis (Patel et al., The Journal of Antibiotics (1995), Vol. 48(9), 997-1003).

According to the invention, it was surprisingly found out by carrying out experiments with knock-out mutants that bacillaene is able to effectively inhibit the growth of commercially relevant pathogens like in particular toxin producing Clostridium perfringens, which is the cause for necrotic enteritis, Vibrio parahaemolyticus, which is the cause of diseases of crustaceans like the Early Mortality Syndrome, and Salmonella enterica, which is one of the main causes of food poisoning. Thus, it turned out that bacteria which produce bacillaene are suitable to be used as probiotic feed and food ingredients and that bacillaene or derivatives thereof are suitable for treating bacterial diseases.

Thus, a first subject of the invention are compositions, in particular food, feed and therapeutic compositions as well as compositions for treating plants, comprising bacillaene or a derivative thereof, in particular bacillaene producing microorganisms and/or bacillaene containing preparations thereof.

According to the invention, “derivative of bacillaene” in particular refers to hydrogenated variants of bacillaene, in particular to dihydrobacillaene as documented in the literature.

Thus, a further subject of the invention is also the use of bacillaene producing microorganisms or preparations thereof as probiotic feed or food ingredients.

A further subject of the invention are in particular also bacillaene producing microorganisms, which are suitable as probiotics. For being suitable as probiotic, the microorganisms according to the invention should preferably fulfil certain criteria like bile resistance, heat resistance and/or ability to grow under anaerobic conditions.

The bacillaene producing bacteria according to the invention are in particular able to inhibit strains selected from Clostridium, preferably C. perfringens, in particular C. perfringens ATCC 13124, Salmonella, preferably Salmonella enterica, in particular Salmonella enterica subsp. enterica enteritidis DSM 14221, E. coli, in particular E. coli ATCC11775, and Vibrio, preferably Vibrio parahaemolyticus, in particular Vibrio parahaemolyticus DSM10027 and/or Vibrio parahaemolyticus TW01.

The bacillaene producing microorganisms according to the invention are preferably bacteria and more preferably selected from Bacillus, in particular B. subtilis, B. amyloliquefaciens, B. methylotrophicus and B. atropheus, and Paenibacillus, in particular P. polymixa and P. durus. In a very preferred embodiment of the invention the bacillaene producing microorganism is a B. amyloliquefaciens or B. subtilis.

The bacillaene producing microorganisms according to the invention are preferably further characterized in that they are able to grow in presence of 2 mM bile, preferably in presence of 4 mM bile, in particular characterized by an AUC5 performance value of at least 0.5, preferably at least 0.65, above all at least 0.8, and an AUC10 performance value of at least 1.2, preferably at least 1.4, above all at least 1.6, in presence of 2 mM bile and/or in that they are able to grow in presence of 0.3 wt.-% bile, in particular in presence of 0.3 wt.-% chicken bile and/or in presence of 0.3 wt.-% porcine bile, preferably characterized by being able to survive exposure to 0.3 wt.-% bile, in particular 0.3 wt.-% chicken bile and/or 0.3 wt.-% porcine bile, for at least 3 hours, preferably for at least 5 or 8 hours.

The bacillaene producing microorganisms according to the invention are preferably further characterized by being able to grow anaerobically, in particular by being able to degrade water-insoluble cellulose and protein under anaerobic conditions.

The bacillaene producing microorganisms according to the invention are preferably further characterized in that at least 50%, preferably at least 70 or 90%, of the spores survive exposure to 99° C. for 20 minutes.

A Bacillus amyloliquefaciens strain which turned out to be a suitable bacillaene producing probiotic has been identified by screening of naturally occurring isolates and has been deposited at the DSMZ (Leibniz-Institute DSMZ-German Collection of Microorganisms and Cell Cultures, Inhoffenstraße 7B, 38124 Braunschweig, Germany) on Feb. 7, 2019 under the provisions of the Budapest Treaty on the International Recognition of the Deposit of Microorganisms for the Purpose of Patent Procedure under the Accession Number DSM 33014 in the name of Evonik Degussa GmbH.

Thus, in a preferred embodiment of the invention, the bacillaene producing microorganism is selected from the following group:
a) The Bacillus amyloliquefaciens strain DSM 33014;
b) a mutant of the Bacillus strain as deposited under DSM 33014 with preferably a sequence identity of at least 98%, in particular 99 or 99.5% to the genome sequence of the Bacillus strain as deposited under DSM 33014, wherein the mutant preferably exhibits bile resistance and/or heat resistance and/or is able to grow under anaerobic conditions.

The B. amyloliquefaciens strain DSM 33014 exhibits a 16 S rDNA according to SEQ ID NO: 1, a yqfD sequence according to SEQ ID NO: 2, a gyrB sequence according to SEQ ID NO: 3, a rpoB sequence according to SEQ ID NO: 4 and a groEL sequence according to SEQ ID NO: 5.

Thus, in a preferred embodiment of the invention, preferred bacillaene producing Bacillus strains, in particular the Bacillus amyloliquefaciens strain DSM 33014, have at least one, preferably at least 3, more preferably all, of the following characteristics:
a) a 16 S rDNA sequence with a sequence identity of at least 98, preferably at least 99, 99.5 or 99.8%, more preferably 100%, to the 16 S rDNA sequence of the strain DSM 33014 and/or to SEQ ID NO: 1;
a) a yqfD sequence with a sequence identity of at least 98, preferably at least 99, 99.5 or 99.8%, more preferably 100%, to the yqfD sequence of the strain DSM 33014 and/or to SEQ ID NO: 2;
b) a gyrB sequence with a sequence identity of at least 98, preferably at least 99, 99.5 or 99.8%, more preferably 100%, to the gyrB sequence of the strain DSM 33014 and/or to SEQ ID NO: 3;
c) a rpoB sequence with a sequence identity of at least 98, preferably at least 99, 99.5 or 99.8%, more preferably 100%, to the rpoB sequence of the strain DSM 33014 and/or to SEQ ID NO: 4;
d) a groEL sequence with a sequence identity of at least 98, preferably at least 99, 99.5 or 99.8%, more preferably 100%, to the groEL sequence of the strain DSM 33014 and/or to SEQ ID NO: 5.

The baciallene producing bacteria according to the invention comprise a gene cluster which allows the production of bacillaene. The gene cluster preferably comprises the genes baeJ, baeL, baeM, baeN and baeR, encoding the subunits of the bacillaene synthesizing protein complex, which is also called polyketide synthase or PKS protein.

The B. amyloliquefaciens strain DSM 33014 exhibits a baeJ sequence according to SEQ ID NO: 6, a baeL sequence according to SEQ ID NO: 7, a baeM sequence according to SEQ ID NO: 8, a baeN sequence according to SEQ ID NO: 9 and a baeR sequence according to SEQ ID NO: 10.

Thus, according to the invention the gene cluster of the PKS protein preferably comprises the following genes:
a) A baeJ gene with a sequence identity of at least 80%, preferably at least 85, 90 or 95%, more preferably at least 98 or 99%, above all 100%, to the sequence of the baeJ sequence of the strain DSM 33014 and/or to SEQ ID NO: 6;
b) A baeL gene with a sequence identity of at least 80%, preferably at least 85, 90 or 95%, more preferably at least 98 or 99%, above all 100%, to the sequence of the baeL sequence of the strain DSM 33014 and/or to SEQ ID NO: 7;
a) A baeM gene with a sequence identity of at least 80%, preferably at least 85, 90 or 95%, more preferably at least 98 or 99%, above all 100%, to the sequence of the baeM sequence of the strain DSM 33014 and/or to SEQ ID NO: 8;
c) A baeN gene with a sequence identity of at least 80%, preferably at least 85, 90 or 95%, more preferably at least 98 or 99%, above all 100%, to the sequence of the baeN sequence of the strain DSM 33014 and/or to SEQ ID NO: 9;
d) A baeR gene with a sequence identity of at least 80%, preferably at least 85, 90 or 95%, more preferably at least 98 or 99%, above all 100%, to the sequence of the baeR sequence of the strain DSM 33014 and/or to SEQ ID NO: 10.

The microorganisms according to the invention are preferably natural isolates, but they may also be mutants of natural isolates, in particular spontaneous mutants. Furthermore, the microorganisms according to the invention may also be obtained by genetic engineering, in particular by incorporating the genes coding for the bacillaene producing enzyme subunits, in particular the genes baeJ, baeL, baeM, baeN and baeR as mentioned before, into a microorganism, preferably into a microorganism as mentioned before, more preferably into a B. subtilis or B. amyloliquefaciens strain, wherein before incorporation of the genes the microorganism is preferably not able to produce bacillaene.

The term “spontaneous mutant” refers to mutants that arise from natural isolates without the intentional use of mutagens. Such spontaneous mutants may be obtained by classical methods, such as growing the natural isolate in the presence of UV light and/or by applying high temperature or protoplast formation and/or in the presence of a certain antibiotic to which the parent strain is susceptible and testing any resistant mutants for improved biological activity or improved ability to enhance one or more of the indicia of animal health, in particular gut health. Other methods for identifying spontaneous mutants are known to those of ordinary skill in the art. But besides these preferred spontaneous mutants all other kinds of mutants of the natural isolates, like mutants obtained by genetic engineering, are also comprised by the invention.

Thus, one particular embodiment of the invention are naturally non-occurring mutants, in particular spontaneous mutants as defined before, of naturally occurring bacillaene producing strains, preferably characterized by the features as mentioned above in the description.

Thus, one further particular embodiment of the invention are bacillaene producing microorganisms, which have been obtained by incorporation of the genes encoding for the subunits of the bacillaene producing enzymes, in particular the genes baeJ, baeL, baeM, baeN and baeR as mentioned before in the description.

In a preferred embodiment of the invention, the microorganisms and preparations of the present invention are administered orally to animals or human beings.

Thus, a further subject of the invention are compositions, such as feedstuffs, foodstuffs, drinking and rearing water as well as therapeutic compositions, containing bacillaene producing microorganisms and/or preparations thereof and/or bacillaene or a derivative thereof.

A further subject of the invention is also the use of bacillaene producing microorganisms and/or a preparations thereof as probiotic ingredients (DFM) in feed or food products.

Preferred foodstuffs according to the invention are dairy products, in particular yoghurt, cheese, milk, butter and quark.

The cells of the microorganisms of the invention may be present, in particular in the compositions of the invention, as spores (which are dormant), as vegetative cells (which are growing), as transition state cells (which are transitioning from growth phase to sporulation phase) or as a combination of at least two, in particular all of these types of cells. In a preferred embodiment, the composition of the invention comprises mainly or only spores.

In addition or as alternative the cells of the microorganisms may also be used in non-living, inactivated form, as also the non-living cells are expected to still have a probiotic effect. Ways to inactivate the cells are known to those skilled in the art.

The bacillaene producing microorganisms of the invention and compositions containing them, when administered to animals, preferably enhance the health of such animals and/or improve the general physical condition of such animals and/or improve the feed conversion rate of such animals and/or decrease the mortality rate of such animals and/or increase the survival rates of such animals and/or improve the weight gain of such animals and/or increase the productivity of such animals and/or increase the disease resistance of such animals and/or increase the immune response of such animals and/or establish or maintain a healthy gut microflora in such animals and/or reduce the pathogen shedding through the feces of such animals. In particular the microorganisms and compositions of the invention might be used to assist in re-establishing a healthy balance of the gut microflora after administration of antibiotics for therapeutic purposes.

A further subject of the invention is therefore a method of enhancing the health of animals and/or of improving the general physical condition of animals and/or of improving the feed conversion rate of animals and/or of decreasing the mortality rate of animals and/or of increasing the survival rates of animals and/or of improving the weight gain of animals and/or of increasing the productivity of animals and/or of increasing the disease resistance of animals and/or of increasing the immune response of animals and/or of establishing or maintaining a healthy gut microflora in animals and/or of reducing the pathogen shedding through the feces of animals, wherein the microorganisms of the invention and/or preparations thereof and/or the compositions of the invention and/or bacillaene or a derivative thereof are administered to animals, in particular to aquatic animals.

A further subject of the invention is therefore also the use of microorganisms and/or preparations and/or compositions of the invention for enhancing the health of animals and/or for improving the general physical condition of animals and/or for improving the feed conversion rate of animals and/or for decreasing the mortality rate of animals and/or for increasing the survival rates of animals and/or for improving the weight gain of animals and/or for increasing the productivity of animals and/or for increasing the disease resistance of animals and/or for increasing the immune response of animals and/or for establishing or maintaining a healthy gut microflora in animals and/or for reducing the pathogen shedding through the feces of animals, wherein the microorganisms and/or preparations and/or compositions of the invention and/or bacillaene or a derivative thereof are administered to animals and wherein the animals are in particular aquatic animals.

A further subject of the invention are therefore also the microorganisms and/or preparations and/or compositions of the invention as mentioned before and/or bacillaene or a derivative thereof for enhancing the health of animals and/or for improving the general physical condition of animals and/or for improving the feed conversion rate of animals and/or for decreasing the mortality rate of animals and/or for increasing the survival rate of animals and/or for improving the weight gain of animals and/or for increasing the productivity of animals and/or for increasing the disease resistance of animals and/or for increasing the immune response of animals and/or for establishing or maintaining a healthy gut microflora in animals and/or for reducing the pathogen shedding through the feces of animals.

“Increasing the productivity of animals” refers in particular to any of the following: production of more or higher quality eggs, milk or meat or increased production of weaned offspring.

The methods and uses of the microorganisms, preparations and compositions of the invention can be therapeutic or non-therapeutic. In a particularly preferred embodiment of the invention, the methods and uses are non-therapeutic, in particular feeding applications.

As the untreated manure of animals due to pathogenic bacteria and other ingredients may have a detrimental environmental effect, in particular with respect to the animals themselves and/or with respect to human beings getting in contact with the manure, which can be avoided by either feeding the animals or directly treating the manure or the bedding of the animals with the microorganisms, compositions or preparations of the invention, therefore a further subject of the invention is a method of controlling and/or avoiding detrimental environmental effects of manure or contaminated liquids, the method comprising the step of applying to manure, contaminated liquids, litter, a pit, or a manure pond at least one bacillaene producing microorganism and/or a preparation thereof and/or a composition according to the invention. Preferably the microorganisms, preparations or compositions are applied in liquid form, for example by spraying, or as a powder, for example by strewing.

As detrimental bacteria may have a negative influence on the consistency of litter and in particular may effect a rather fluid or highly fluid litter, which might lead to foot pad lesions of poultry and which can be avoided by feeding the animals with the microorganisms, compositions or preparations of the invention, therefore a further subject of the invention is a method of controlling and/or improving the consistency of litter, in particular a method of ensuring a solid consistency of litter and/or a method of avoiding foot pad lesions, the method comprising the step of feeding animals, in particular poultry, with at least one microorganism, one preparation and/or one composition according to the invention.

The microorganisms and preparations according to the invention can also be used for improving the quality of feed and food compositions as well as for improving the quality of water and aqueous solutions. A further subject of the invention is therefore also a method of controlling and/or improving the quality of feed or food compositions as well as the quality of water or aqueous solutions, in particular of drinking water and/or rearing water, comprising the step of applying to feed, food, water or an aqueous solution at least one microorganism and/or at least one preparation and/or at least one composition of the invention.

Further, the microorganisms and preparations according to the invention can also be used for treating microbial diseases of plants. A further subject of the invention is therefore also a method of treating and/or preventing microbial diseases of plants, in particular of cultivated plants, comprising the step of applying to the plants at least one microorganism and/or at least one preparation and/or at least one composition of the invention. The application may be carried out in liquid form, such as by spraying, or in solid form, in particular as a powder, preferably as a formulated powder.

By using the microorganisms, preparations and compositions of the invention preferably an improvement of at least one of the features as mentioned before is realized, wherein realization of the feature preferably means an improvement of at least 1%, more preferably of at least 3 or at least 5%, in comparison to an adequate negative control. As negative control averages known in the animal husbandry field may be used, but preferably as negative control animals which are subjected to the same treatment like the animals tested are used, but without administration of the microorganisms and/or preparations of the invention.

Besides their ability, due to the production of bacillaene, to inhibit the growth of the pathogenic bacteria as mentioned before, the microorganisms and preparations as used according to the invention are preferably able to inhibit the growth of further pathogenic bacteria.

In particular, the microorganisms, preparations and compositions of the invention may be administered or fed to an animal in an amount effective to inhibit and/or decrease the growth of pathogenic bacteria in the animal gut. Such pathogenic bacteria include besides Clostridia, Salmonella, Vibrio and Escherichia coli in particular also Listeria, Enterococci, Staphylococci, Aeromonas, Streptococci, Campylobacter, Shigella, Haemophilus and Brachyspira. Relatedly, the methods of the present invention may be used to decrease the amount of pathogenic bacteria, viruses and protozoans shed in animal feces. The methods of the present invention may also be used to maintain or increase the growth of beneficial bacteria, such as lactic acid bacteria, in the animal gut. By decreasing pathogenic bacteria and/or increasing or maintaining beneficial bacteria, the compositions of the present invention are able to maintain an overall healthy gut microflora.

Thus, a further subject of the invention is also a method of inhibiting and/or decreasing the growth of pathogenic bacteria and/or for maintaining and/or increasing the growth of beneficial bacteria, in particular in an animal or human gut, wherein the microorganisms, preparations and/or compositions of the invention are administered to animals or humans, and wherein the pathogenic bacteria are preferably selected from Clostridia, in particular from C. perfringens, C. difficile, C. novyi, C. septicum and C. colinum, from Listeria, in particular from L. monocytogenes, L. seeligeri and L. welshimeri, from Salmonella, in particular S. enterica including the subspecies enterica, arizonae, bongori and in particular the serovars, S. gallinarum, S. pullorum, S. typhimurium, S. enteritidis, S. cholerasuis, S. heidelberg, S. dublin, S. hadar, S. typhi, S. paratyphi and S. infantis, from Enterococci, in particular E. faecalis, E. faecium and E. cecorum, from Staphylococci, in particular S. aureus, from Aeromonas, from Streptococci, in particular S. suis and S. gallinaceus, from Campylobacter, in particular C. jejuni and C. coli, from Escherichia coli, from Haemophilus, in particular Haemophilus parasuis, from Brachyspira, in particular Brachyspira hyodysenteriae and from Vibrio, in particular V. parahaemolyticus and V. harveyi, and the beneficial bacteria are preferably selected from lactic acid bacteria, in particular from lactobacilli and bifidobacteria. The pathogenic microorganisms are selected in a preferred embodiment from Clostridia, in particular from C. perfringens, Salmonella, in particular S. enterica, and Vibrio, in particular V. parahaemolyticus.

In a preferred embodiment of the invention the amount of at least one pathogenic bacterium, in particular the amount of C. perfringens, S. enterica and/or V. parahaemolyticus, is reduced by at least 0.5 log, more preferably by at least 1 log, 2 log, or 3 log.

Thus, a further subject of the invention are also the microorganisms, preparations and compositions of the invention for inhibiting and/or decreasing the growth of pathogenic bacteria and/or for maintaining and/or increasing the growth of beneficial bacteria, in particular in an animal or human gut, wherein the pathogenic bacteria are preferably selected from Clostridia, in particular from C. perfringens, C. difficile, C. novyi, C. septicum and C. colinum, from Listeria, in particular from L. monocytogenes, L. seeligeri and L. welshimeri, from Salmonella, in particular S. enterica including the subspecies enterica, arizonae, bongori and in particular the serovars, S. gallinarum, S. pullorum, S. typhimurium, S. enteritidis, S. cholerasuis, S. heidelberg, S. dublin, S. hadar, S. typhi, S. paratyphi and S. infantis, from Enterococci, in particular E. faecalis, E. faecium and E. cecorum, from Staphylococci, in particular S. aureus, from Aeromonas, from Streptococci, in particular S. suis and S. gallinaceus, from Campylobacter, in particular C. jejuni and C. coli, from Escherichia coli, from Haemophilus, in particular Haemophilus parasuis, from Brachyspira, in particular Brachyspira hyodysenteriae and from Vibrio, in particular V. parahemolyticus and V. harveyi, and the beneficial bacteria are preferably selected from lactic acid bacteria, in particular from lactobacilli and bifidobacteria. The pathogenic microorganisms are selected in a preferred embodiment from Clostridia, in particular from C. perfringens, Salmonella, in particular S. enterica, and Vibrio, in particular V. parahaemolyticus.

The occurrence and/or increased growth of the pathogenic bacteria does or can lead to the outbreak of certain diseases. For example the occurrence and/or increased growth of Clostridium perfringens can lead to the outbreak of gut diseases, in particular to the outbreak of necrotic enteritis in swine and poultry. The occurrence and/or increased growth of C. perfringens can also lead to the outbreak of further diseases like bacterial enteritis, gangrenous dermatitis and colangiohepatitis. Even the mildest form of infection by C. perfringens can already be accompanied by diarrhea, which results in wet litter and by that may lead to secondary diseases like foot pad dermatitis. While C. perfringens type C generally is considered to be the primary cause of necrotic enteritis and necrohemorrhagic enteritis in piglets, type A has been linked to enteric disease in suckling and feeding pigs with mild necrotic enterocolitis and villous atrophy.

Clostridium difficile is an important emerging pathogen that causes diarrhea primarily in neonatal swine. Affected piglets may have dyspnea, abdominal distention, and scrotal edema.

Staphylococcus aureus subsp. aureus can cause bumblefoot in chickens, streptococcal mastitis in sows and it is capable of generating toxins that produce food poisoning in the human body.

E. cecorum is known to cause lameness, arthritis and osteomyelitis in broilers usually caused by an inflammation of a joint and/or bone tissue. Further E. cecorum can cause an inflammation of the pericardium.

Salmonella bacteria are an important cause of food poisoning of humans, which often is linked to the consumption of meat, such as poultry meat, pork or products derived therefrom. Accordingly, controlling Salmonella is a significant challenge for the meat-producing industry. In Europe, a baseline study conducted in 2005 on the prevalence of Salmonella in egg-laying flocks has shown that at the global EU-level, 20.3% of the large-scale laying hen holdings are bacteriologically positive for S. enteritidis. In some countries, the prevalence was even higher than 80% [European Food Safety Authority (2006), “Preliminary report: analysis of the baseline study on the prevalence of salmonella in laying hen flocks of Gallus gallus”].

S. gallinarum is the cause of the fowl typhoid disease and S. pullorum the cause of the pullorum disease, both diseases which cause big damages in the poultry industry.

C. coli is a foodborne bacterium, most people usually get infected by eating pig meat that contained the bacteria. It causes gastroenteritis and acute enterocolitis in humans, and also of acute diarrheal illnesses. Pigs are the main host, but it can also infect humans, avian species and a wide range of other animals.

S. gallinaceus can cause septicaemia in poultry. The gross lesions included splenomegaly, hepatomegaly, renomegaly and congestion. Multiple areas of necrosis and/or infarction in the liver and spleen associated with valvular endocarditis were also observed.

S. suis is an important pathogen in pigs and one of the most important causes of bacterial mortality in piglets after weaning causing septicemia, meningitis and many other infections.

Vibrio parahaemolyticus is in particular responsible for diseases of crustaceans like the Early Mortality Syndrome (EMS), also known as Acute Hepatopancreatic Necrosis Disease (AHPND), which affects both Giant Tiger Prawn (Penaeus monodon) and Whiteleg Shrimp (Penaeus vannamei).

Pathogens can cause further diseases like polyarthritis, fibrinous polyserositis, post-weaning enteric disorders like post-weaning diarrhea and edema disease and swine dysentery.

A further subject of the invention is therefore also a therapeutic composition comprising the microorganisms and/or preparations and/or compositions of the invention and/or bacillaene or a derivative thereof, in particular a therapeutic composition for treating aquatic animals.

A preferred subject of the invention is therefore a therapeutic composition for treating a disease, in particular gut disease, in particular related to bacterial infection by Clostridia, in particular C. perfringens, and/or by Salmonella, in particular S. enterica, preferably S. enterica subsp. enterica enteritidis, and/or by Vibrio, in particular Vibrio parahaemolyticus.

A further preferred subject in this context is therefore a therapeutic composition for treating and/or preventiing necrotic enteritis and/or necrohemorrhagic enteritis, in particular sub-clinical necrotic enteritis and/or necrohemorrhagic enteritis, in animals, preferably swine or poultry, comprising the strains and/or preparations and/or compositions of the invention and/or bacillaene or a derivative thereof.

A further preferred subject in this context is therefore a therapeutic composition for treating and/or preventing the Early Mortality Syndrome in animals, preferably animals kept in aquaculture, more preferably crustaceans, in particular shrimps and prawns, comprising the microorganisms and/or preparations and/or compositions of the invention and/or bacillaene or a derivative thereof.

A further preferred subject in this context is therefore a therapeutic composition for treating and/or preventing diseases caused by food poisoning, comprising the microorganisms and/or preparations and/or compositions of the invention and/or bacillaene or a derivative thereof.

Another preferred subject in this context is therefore a therapeutic composition for treating and/or preventing of bacterial enteritis, gangrenous dermatitis, colangiohepatitis, clostridiosis, diarrhea, dyspnea, abdominal distention, scrotal edema, bumblefoot, foot pad dermatitis, streptococcal mastitis, lameness, arthritis, polyarthritis, fibrinous polyserositis, post-weaning enteric disorders like post-weaning diarrhea and edema disease, dysentery, osteomyelitis, inflammation of joints and/or bone tissue, inflammation of the pericardium, splenomegaly, hepatomegaly, renomegaly, congestion, necrosis, infarction in the liver or spleen, valvular endocarditis, septicemia and/or meningitis, in animals, preferably in swine or poultry, comprising the microorganisms and/or preparations and/or compositions of the invention and/or bacillaene or a derivative thereof.

A further subject of the invention is therefore also the treatment and/or prevention of a disease, in particular of a gut disease, related to bacterial infection by Clostridia, in particular C. perfringens, and/or by Salmonella, in particular S. enterica, preferably S. enterica subsp. enterica enteritidis, and/or by Vibrio, in particular Vibrio parahaemolyticus, wherein a microorganism and/or preparation and/or composition of the invention and/or bacillaene or a derivative thereof is administered to an animal in need thereof, wherein the animal is preferably swine or poultry.

A further subject of the invention is therefore also the treatment and/or prevention of a disease, in particular of a gut disease, preferably of necrotic enteritis or necrohemorrhagic enteritis, in particular of sub-clinical necrotic enteritis or sub-clinical necrohemorrhagic enteritis, wherein a microorganism and/or preparation and/or composition of the invention and/or bacillaene or a derivative thereof is administered to an animal in need thereof, wherein the animal is preferably swine or poultry.

A further subject of the invention is therefore also the treatment and/or prevention of a disease of aquatic animals, in particular of the Early Mortality Syndrome, wherein a microorganism and/or preparation and/or composition of the invention and/or bacillaene or a derivative thereof is administered to an animal in need thereof, wherein the animal is preferably an animal kept in aquaculture, more preferably crustaceans, in particular shrimps and prawns.

A further subject of the invention is therefore also the treatment and/or prevention of diseases caused by food poisoning, wherein a microorganism and/or preparation and/or composition of the invention and/or bacillaene or a derivative thereof is administered to an animal or human being in need thereof.

A further subject of the invention is therefore also the treatment and/or prevention of diseases caused by food poisoning, wherein a microorganism and/or preparation and/or composition of the invention and/or bacillaene or a derivative thereof is applied to an animal product, in particular to meat or eggs, to avoid the poisoning of human beings.

A further subject of the invention is therefore also the treatment and/or prevention of a disease, preferably a disease of swine or poultry, selected from bacterial enteritis, gangrenous dermatitis, colangiohepatitis, clostridiosis, diarrhea, dyspnea, abdominal distention, scrotal edema, bumblefoot, foot pad dermatitis, streptococcal mastitis, lameness, arthritis, polyarthritis, fibrinous polyserositis, post-weaning enteric disorders like post-weaning diarrhea and edema disease, dysentery, osteomyelitis, inflammation of joints and/or bone tissue, inflammation of the pericardium, splenomegaly, hepatomegaly, renomegaly, congestion, necrosis, infarction in the liver or spleen, valvular endocarditis, septicemia and/or meningitis, wherein a microorganism and/or preparation and/or composition of the invention and/or bacillaene or a derivative thereof is administered to an animal in need thereof.

The microorganisms and/or preparations and/or compositions of the invention can be administered to animals in feed and/or drinking water over multiple days throughout the animal's life or during particular stages or portions of the animal's life. For example, the microorganisms and/or preparations and/or compositions can be administered only in a starter diet or only in a finisher diet of farm animals.

A particular subject of the invention is also a method of enhancing the health of human beings and/or of improving the general physical condition of human beings and/or of increasing the disease resistance of human beings and/or of increasing the immune response of human beings and/or of establishing or maintaining a healthy gut microflora in human beings, wherein the microorganisms and/or preparations and/or compositions of the invention and/or bacillaene or a derivative thereof are administered to human beings.

A further subject of the invention is therefore also the use of microorganisms and/or preparations and/or compositions of the invention for enhancing the health of human beings and/or for improving the general physical condition of human beings and/or for increasing the disease resistance of human beings and/or for increasing the immune response of human beings and/or for establishing or maintaining a healthy gut microflora in human beings, wherein the microorganisms and/or preparations and/or compositions of the invention and/or bacillaene or a derivative thereof are administered to human beings.

The compositions of the present invention, in particular the feed, food and pharmaceutical compositions as well as the drinking or rearing water, preferably comprise the microorganisms of the invention and are administered to animals at a rate of about 1×10³ to about 2×10¹² CFU/g feed or ml water, in particular in a rate of about 1×10³ or about 1×10⁴ or about 1×10⁵ or about 1×10⁸ or about 1×10⁷ or about 1×10⁸ or about 1×10⁹ or about 1×10¹⁰ or about 1×10¹¹ or about 1×10¹² CFU/g feed or ml water, preferably in an amount of about 1×10⁴ to about 1×10¹⁰ CFU/g feed or ml water, more preferably in an amount of 1×10⁴ to 1×10⁷ CFU/g feed or ml water.

Correspondingly, preferred amounts of the strains and/or preparations of the invention in the feed, food and water compositions of the invention range preferably from 0.1 wt.-% to 10 wt.%, more preferably from 0.2 wt.-% to 5 wt.-%, in particular from 0.3 wt.-% to 3 wt.-%.

The methods of the present invention may be used for all kinds of animals, in particular all kinds of non-human and non-insect animals, more preferably all kinds of vertebrates such as mammals, aquatic animals and birds.

Animals that may benefit from the invention include but are not limited to farm animals, pets, exotic animals, zoo animals, aquatic animals, animals used for sports, recreation or work.

Pets are preferably selected from dogs, cats, domestic birds and domestic exotic animals.

Aquatic animals are preferably selected from finfish and crustaceans which are preferably intended for human nutrition. These include, in particular, carp, tilapia, catfish, tuna, salmon, trout, barramundi, bream, perch, cod, shrimps, lobster, crabs, prawns and crayfish. Preferred types of salmon in this context are the Atlantic salmon, red salmon, masu salmon, king salmon, keta salmon, coho salmon, Danube salmon, Pacific salmon and pink salmon.

Further preferred aquatic animals are farming fish which are subsequently processed to give fish meal or fish oil. In this connection, the fish are preferably herring, pollack, menhaden, anchovies, capelin or cod.

In a further preferred embodiment, the animals are farm animals, which are raised for consumption or as food-producers, such as poultry, swine and ruminants.

The poultry may be selected from productive or domestic poultry, but also from fancy poultry or wild fowl.

Preferred productive poultry in this context are chickens, turkeys, ducks and geese. The productive livestock in this context is preferably poultry optimized for producing young stock or poultry optimized for bearing meat.

Preferred fancy poultry or wild fowl are peacocks, pheasants, partridges, chukkars, guinea fowl, quails, capercaillies, grouse, pigeons and swans, with quails being especially preferred.

Further preferred poultry are ratites, in particular ostriches and emus, as well as parrots.

Ruminants according to the invention are preferably selected from cattle, goat and sheep. In one embodiment, the compositions of this invention may be fed to preruminants to enhance their health and, in particular, to decrease the incidence of diarrhea in these animals. Preruminants are ruminants, including calves, ranging in age from birth to about twelve weeks.

A particularly preferred embodiment of the invention is therefore a method of feeding animals, preferably terrestrial animals, in particular swine or avian, or aquatic animals, in particular crustaceans like shrimps and prawns, comprising administering to the animals a bacillaene producing microorganism or a preparation thereof or a feed composition containing the bacillaene producing microorganisms or preparation thereof,

The compositions of the invention may comprise at least one carrier or typical feed ingredient or combinations thereof.

Suitable carriers are inert formulation ingredients added to improve recovery, efficacy, or physical properties and/or to aid in packaging and administration. Such carriers may be added individually or in combination. These carriers may be selected from anti-caking agents, anti-oxidation agents, bulking agents, and/or protectants. Examples of useful carriers include polysaccharides (in particular starches, maltodextrins, methylcelluloses, gums, chitosan and/or inulins), protein sources (in particular skim-milk powder and/or sweet-whey powder), peptides, sugars (in particular lactose, trehalose, sucrose and/or dextrose), lipids (in particular lecithin, vegetable oils and/or mineral oils), salts (in particular sodium chloride, sodium carbonate, calcium carbonate, chalk, limestone, magnesium carbonate, sodium phosphate, calcium phosphate, magnesium phosphate and/or sodium citrate), and silicates (in particular clays, in particular beolite clay, amorphous silica, fumed/precipitated silicas, zeolites, Fuller's earth, baylith, clintpolite, montmorillonite, diatomaceous earth, talc, bentonites, and/or silicate salts like aluminium, magnesium and/or calcium silicate). Suitable carriers for animal feed additives are set forth in the American Feed Control Officials, Inc.'s Official Publication, which publishes annually. See, for example Official Publication of American Feed Control Officials, Sharon Krebs, editor, 2006 edition, ISBN 1-878341-18-9. The carriers can be added after concentrating the fermentation broth and/or during and/or after drying. Preferred carriers according to the invention are selected from calcium carbonate, diatomaceous earth and vegetable oil.

A preferred embodiment of the invention are concentrate compositions, in particular feed additive compositions, i.e. compositions suitable for preparing a feed composition, which comprise at least one microorganism of the invention and at least one carrier as mentioned before, wherein the at least one microorganism is preferably comprised in an amount of 0.1 to 10 wt.-%, more preferably in an amount of 0.2 to 5 wt.-%, in particular in an amount of 0.3 to 3 wt.-%, above all in an amount of 0.4 to 2.2 wt.-%, and the at least one carrier is preferably comprised in an amount of at least 90 wt.-%, preferably in an amount of 90 to 99.9 wt.-%, more preferably in an amount of 95 to 99.8 wt.-%, in particular in an amount of 97 to 99.7 wt.-%, above all in an amount of 97.8 to 99.6 wt.-%, and wherein the carrier consists preferably substantially of limestone, in particular of limestone with smaller parts of diatomaceous earth and/or vegetable oil.

These preferred compositions of the invention, which contain stabilized microorganisms, can be used for the preparation of feed and pharmaceutical compositions as well as drinking and rearing water which preferably comprise the strains according to the invention in an amount as mentioned in the specification above. In a preferred embodiment 50 to 1000 grams of such a concentrate composition, in particular 50, 100, 250, 500 or 1000 grams of such a concentrate composition, are used per ton of feed, drinking or rearing water to provide compositions which can be used for feeding animals. These concentrate compositions preferably comprise at least one strain of the invention in an amount of 1×10⁹ to 2×10¹¹ CFU, in particular 2×10⁹ to 1×10¹¹ CFU, per g of the concentrate composition.

Starting from these concentrate compositions, feed and food compositions can be prepared by mixing the concentrate compositions with typical feed or food ingredients, respectively.

Suitable typical animal feed ingredients which may be contained in the compositions according to the invention and/or used in the preparation of feed compositions starting from concentrate compositions according to the invention include one or more of the following: proteins, carbohydrates, fats, further probiotics, prebiotics, enzymes, vitamins, immune modulators, milk replacers, minerals, amino acids, coccidiostats, acid-based products and/or medicines, such as antibiotics.

Carbohydrates containing components which may be used according to the invention are for example forage, roughage, wheat meal, sunflower meal or soya meal, and mixtures thereof.

Proteins containing components which may be used according to the invention are for example soya protein, pea protein, wheat gluten or corn gluten, and mixtures thereof.

Fats containing components which may be used according to the invention are in particular oils, of both animal and plant origin, like vegetable oils, for example soya bean oil, rapeseed oil, sunflower seed oil, flaxseed oil or palm oil, fish oil, and mixtures thereof.

Proteins containing components which additionally contain fats which may be used according to the invention are for example fish meal, krill meal, bivalve meal, squid meal or shrimp shells, as well as combinations thereof.

Further probiotics (DFM) which may be used according to the invention in combination with the microorganisms and preparations of the invention are preferably bacteria selected from the species Bacillus subtilis, Bacillus licheniformis, Bacillus lentus, Bacillus pumilus, Bacillus laterosporus, Bacillus coagulans, Bacillus alevi, Bacillus cereus, Bacillus badius, Bacillus thurigiensis, Enterococcus faecium, and Pediococcus acidilactici. Preferred examples are Bacillus subtilis DSM 32539 (as deposited with the DSMZ on Jun. 14, 2017 under the provisions of the Budapest Treaty on the International Recognition of the Deposit of Microorganisms for the Purpose of Patent Procedure) and derivatives thereof, Bacillus licheniformis DSM 32314 and Bacillus subtilis DSM 32315 (both deposited with the DSMZ on May 12, 2016 under the provisions of the Budapest Treaty on the International Recognition of the Deposit of Microorganisms for the Purpose of Patent Procedure) and derivatives thereof, Bacillus subtilis PB6 (as described in U.S. Pat. No. 7,247,299 and deposited as ATCC Accession No. PTA-6737), which is sold by Kemin under the trademark CLOSTAT®, Bacillus subtilis C-3102 (as described in U.S. Pat. No. 4,919,936 and deposited as FERM BP-1096 with the Fermentation Research Institute, Agency of Industrial Science and Technology, in Japan), sold by Calpis as CALSPORIN®, Bacillus subtilis DSM 17299, as sold by Chr. Hansen under the trademark GalliPro®, Bacillus licheniformis DSM 17236, as sold by Chr. Hansen under the trademark GalliProTect®, a mixture of Bacillus licheniformis DSMZ 5749 and Bacillus subtilis DSMZ 5750 spores, as sold by Chr. Hansen under the trademark BioPlus®YC, B. subtilis DSM 29784, as sold by Adisseo/Novozymes under the trademark Alterion®, Bacillus subtilis, as sold by Chr. Hansen under the trademark PORCBOOST®, or Bacillus coagulans strains as described in U.S. Pat. No. 6,849,256. Other non-Bacillus probiotics, such as Saccharomyces cerevisiae, Pichia pastoris, Aspergillus niger, Aspergillus oryzae, or Hansenula, may also be used in compositions of the present invention. In particular in food compositions further probiotics which are known to be useful to the human health may be used such as lactic acid producing bacteria, in particular lactobacilli, or Bifidobacteria. If said further probiotics are not formulated as part of the compositions of the present invention, they may be administered together (either at the same time or at different times) with the compositions of the present invention.

Prebiotics which may be used according to the invention are preferably oligosaccharides, in particular selected from galactooligosaccharides, silayloligosaccharides, lactulose, lactosucrose, fructooligosaccharides, palatinose or isomaltose oligosaccharides, glycosyl sucrose, maltooligosaccharides, isomaltooligosaccharides, cyclodextrins, gentiooligosaccharides, soybean oligosaccharides, xylooligosaccharides, dextrans, pectins, polygalacturonan, rhamnogalacturonan, mannan, hemicellulose, arabinogalactan, arabinan, arabinoxylan, resistant starch, mehbiose, chitosan, agarose, inulin, tagatose, polydextrose, and alginate.

Enzymes which may be used in feed compositions according to the invention and which may aid in the digestion of feed, are preferably selected from phytases (EC 3.1.3.8 or 3.1.3.26), xylanases (EC 3.2.1.8), galactanases (EC 3.2.1.89), galactosidases, in particular alpha-galactosidases (EC 3.2.1.22), proteases (EC 3.4), phospholipases, in particular phospholipases A1 (EC 3.1.1.32), A2 (EC 3.1.1.4), C (EC 3.1.4.3), and D (EC 3.1.4.4), lysophospholipases (EC 3.1.1.5), amylases, in particular alpha-amylases (EC 3.2.1.1); lysozymes (EC 3.2.1.17), glucanases, in particular beta-glucanases (EC 3.2.1.4 or EC 3.2.1.6), glucoamylases, cellulases, pectinases, or any mixture thereof.

Examples of commercially available phytases include Bio-Feed™ Phytase (Novozymes), Ronozyme® P and HiPhos™ (DSM Nutritional Products), Natuphos™ (BASF), Finase® and Quantum® Blue (AB Enzymes), the Phyzyme® XP (Verenium/DuPont) and Axtra® PHY (DuPont). Other preferred phytases include those described in e.g. WO 98/28408, WO 00/43503, and WO 03/066847.

Examples of commercially available xylanases include Ronozyme® WX and G2 (DSM Nutritional Products), Econase® XT and Barley (AB Vista), Xylathin® (Verenium) and Axtra® XB (Xylanase/beta-glucanase, DuPont). Examples of commercially available proteases include Ronozyme® ProAct (DSM Nutritional Products).

Vitamins which may be used according to the invention are for example vitamin A, vitamin D3, vitamin E, vitamin K, e.g., vitamin K3, vitamin B12, biotin, choline, vitamin B1, vitamin B2, vitamin B6, niacin, folic acid and panthothenate, e.g. , Ca-D-panthothenate, or combinations thereof.

Immune modulators which may be used are for example antibodies, cytokines, spray-dried plasma, interleukins, or interferons, or combinations thereof.

Minerals which may be used according to the invention are for example boron, cobalt, chloride, chromium, copper, fluoride, iodine, iron, manganese, molybdenum, selenium, zinc, calcium, magnesium, potassium, or sodium, or combinations thereof.

Amino acids which may be used according to the invention are for example lysine, alanine, threonine, methionine or tryptophan, or combinations thereof.

Thus, a further embodiment of the invention is a method of preparing an animal feed composition comprising mixing at least one microorganism and/or at least one preparation and/or at least one concentrate composition of the invention, in particular in an amount effective to enhance animal health, in particular gut health, with feed ingredients, such as proteins, lipids and/or carbohydrates, and optionally further beneficial substances, preferably as mentioned before, to provide a feeding product. This method may comprise for example also a pelleting step.

Standard pelleting processes known to those of skill in the art may be used, including extrusion processing of dry or semi-moist feeds. Preferred pelleting temperatures are between about 65° C. and about 120° C.

The microorganisms and compositions of the present invention can be obtained by culturing the microorganisms of the invention according to methods well known in the art, including by using the media and other methods as described for example in U.S. Pat. No. 6,060,051, EP0287699 or US2014/0010792. Conventional large-scale microbial culture processes include submerged fermentation, solid state fermentation, or liquid surface culture. Towards the end of fermentation, as nutrients are depleted, the cells begin the transition from growth phase to sporulation phase, such that the final product of fermentation is largely spores, metabolites and residual fermentation medium. Sporulation is part of the natural life cycle of these microorganisms and is generally initiated by the cell in response to nutrient limitation. Fermentation is configured to obtain high levels of colony forming units of the cells and to promote sporulation. The bacterial cells, spores and metabolites in culture media resulting from fermentation may be used directly or concentrated by conventional industrial methods, such as centrifugation, tangential-flow filtration, depth filtration, and evaporation. The concentrated fermentation broth may be washed, for example via a diafiltration process, to remove residual fermentation broth and metabolites.

The fermentation broth or broth concentrate can be dried with or without the addition of carriers using conventional drying processes or methods such as spray drying, freeze drying, tray drying, fluidized-bed drying, drum drying, or evaporation. The resulting dry products may be further processed, such as by milling or granulation, to achieve a specific particle size or physical format. Carriers, as described above, may also be added post-drying.

Preparations of the microorganisms of the invention, which are a particular subject of the invention, may be cell-free preparations or preparations containing cell debris or preparations containing a mixture of intact cells and cell debris. Particular examples for preparations of the microorganisms are the supernatant of the fermentation broth, as obtained after finishing the fermentation, as well as cytosol preparations which can be obtained by breaking the microbial cells.

Cell-free preparations of the microorganisms of the invention can be obtained for example by centrifugation and/or filtration of the fermentation broth and/or by centrifugation and/or filtration of the suspension as obtained after breaking the microbial cells. Depending on the technique used, these cell-free preparations may not be completely devoid of cells, but may still comprise a smaller amount of cells or cell debris. As the cells secret compounds like metabolites, enzymes and/or peptides into the surrounding medium, the supernatant of the cells comprises a mixture of such compounds, in particular metabolites, enzymes and/or peptides, as secreted by the cells. Thus, in a preferred embodiment of the invention, the preparation of the microorganisms is a supernatant of the fermentation broth.

Compositions comprising cell debris of the microorganisms may be obtained by rupturing the cells applying techniques as known to those of skill in the art, for example by mechanical means or by applying high pressure. Depending on the degree of force applied, a composition comprising only ruptured cells or a composition comprising a mixture of cell debris and intact cells is obtained. Homogenization of the cells may be realized for example by utilizing a French cell press, sonicator, homogenizer, microfluidizer, ball mill, rod mill, pebble mill, bead mill, high pressure grinding roll, vertical shaft impactor, industrial blender, high shear mixer, paddle mixer, and/or polytron homogenizer. Suitable alternatives are enzymatic and/or chemical treatment of the cells.

Cell-free preparations of the invention comprise also preparations which are obtained by first rupturing the cells by applying techniques as mentioned before and subsequently removing the cell debris and the remaining intact cells. Removing of the cell debris and remaining intact cells can be carried out in particular by centrifugation and/or filtration.

The preparations of the microorganisms of the invention may comprise besides bacillaene as active compounds at least one further metabolite, preferably a mixture of further metabolites, as further described below, and/or at least one enzyme selected from proteases, in particular subtilisin, xylanases and/or cellulases, and/or at least one peptide, and/or combinations thereof.

A preparation containing an effective mixture of metabolites as contained in the microorganisms of the invention and/or as contained in the cell preparations as mentioned before, can be obtained for example according to the methods set forth in U.S. Pat. No. 6,060,051. In particular the preparation can be obtained by precipitating the metabolites as contained in the preparations mentioned before by using organic solvents like ethyl acetate and subsequent redissolving of the precipitated metabolites in an appropriate solvent. The metabolites may subsequently be purified by size exclusion filtration that groups metabolites into different fractions based on molecular weight cut-off.

The preparation containing bacillaene and an effective mixture of further metabolites of the invention preferably comprises at least three, more preferably at least 4, 5, 6, 8, 10 or 12, in particular all metabolites of the microorgansims of the invention. The metabolites possess preferably a molecular weight of between 400 and 4000 Dalton, more preferably of between 500 and 3500 Dalton.

Preferably according to the invention always an effective amount of the microorganisms and/or preparations and/or compositions of the invention and/or bacillaene or a derivative thereof is used in the embodiments of the invention. The term “effective amount” refers to an amount which effects at least one beneficial effect to an animal and/or to the environment, in particular with respect to the features as already mentioned before, in comparison to an animal that has not been administered the strains and/or preparations and/or compositions of the invention, but besides that has been administered the same diet (including feed and other compounds).

In case of therapeutic applications preferably a therapeutic amount of the microorganisms and/or preparations and/or compositions of the invention is used. The term “therapeutic amount” refers to an amount sufficient to ameliorate, reverse or prevent a disease state in an animal. Optimal dosage levels for various animals can easily be determined by those skilled in the art, by evaluating, among other things, the composition's ability to (i) inhibit or reduce pathogenic bacteria in the gut at various doses, (ii) increase or maintain levels of beneficial bacteria and/or (iii) enhance animal health, in particular gut health, at various doses.

### WORKING EXAMPLES

### Example 1

### Assessment of Pathogen Inhibition Capacity of Probiotic B. Amyloliquefaciens Strains encoding the Cluster for Bacillaene Biosynthesis

The potential effect of bacillaene on pathogen inhibition by probiotic Bacillus strains was assessed using well diffusion antagonism tests (Parente et al. 1995). Strain DSM 33014 and a further probiotic bacillaene producing B. amyloliquefaciens strain (“B. amyloliquefaciens strain A”) were used in the tests. For B. amyloliquefaciens strain A a knockout-mutant was constructed in which the baeJ gene was disrupted, rendering the strain unable to produce bacillaene (Chen et al., 2006). In addition a B. subtilis strain (“B. subtilis strain B”) was tested, which does not harbor the bacillaene gene cluster.

A well diffusion antagonism test with different pathogens, S. enterica subsp. enterica enteritidis DSM 14221, E. coli ATCC 11775, Vibrio parahaemolyticus DSM 10027 and C. perfringens ATCC 13124 was performed.

Bacillus strains were grown in 10 ml LB Kelly medium containing, per liter, 40 g soy peptone, 40 g dextrin 10, 1.8 g KH2PO4, 4.5 g K2HPO4, 0.3 g MgSO4. 7H2O, and 0.2 ml KellyT trace metal solution (as described in Scholz et al., 2011) for 16 h at 37° C. and 200 rpm in 100 mL shaking flask. The pathogenic strains were grown under suitable conditions as liquid culture to an optical density of 600 nm of at least 1, then 130 μl were spread with a sterile spatula on the surface of agar plates. For all pathogens TSBYE (30 g/l TSB+6 g/l Yeast extract) agar plates were used. 9 mm diameter wells were cut into the dried plates. The first well was used as non-inoculated media control without culture, the other wells were inoculated with 100 μL of a Bacillus culture adjusted to OD₆₀₀ 5. For the assays with E. coli ATCC 11775 and S. enterica subsp. enterica enteritidis DSM 14221 the plates were incubated after incubation for 24 hrs under anaerobic conditions for additional 16 hrs under aerobic conditions. Each plate was analyzed for the appearance of inhibition halos around the cut well. The zone of clearance in mm was determined measuring from the edge of the cut well to the border of the cleared lawn. Each halo was measured twice (horizontally, vertically), then averaged. The results can be found in the following tables 1 and 2. For the assay with V. parahaemolyticus DSM 10027 the Bacillus cultures were only grown for 10 hrs in LB Kelly medium, the OD₆₀₀ was adjusted to an OD₆₀₀ of 10 and the agar plates were directly analyzed after 24 hrs of incubation under anaerobic conditions (see tab. 3). For the inhibition assay with C. perfringens ATCC 13124 on agar plates the Bacillus cultures were grown for 16 hrs, the OD₆₀₀ was adjusted to an OD₆₀₀ of 5 and the agar plates were directly analyzed after 24 hrs of incubation under anaerobic conditions (tab. 4).

TABLE 1
Comparison of B. amyloliquefaciens strains encoding for the bacillaene cluster, a baeJ
knockout mutant and a B. subtilis strain not harboring the bacillaene cluster in its genome
regarding the inhibitory capacity on a pathogenic S. enterica subsp. enterica enteritidis DSM 14221
strain in a well diffusion antagonism assays on TSBYE medium, values in mm clearance of
pathogen.

| Pathogen → | S. enterica subsp. enterica |
|---|---|
| | enteritidis DSM 14221 |
| strain ↓ | [mm] |
| B. amyloliquefaciens DSM 33014 | 21.60 |
| B. amyloliquefaciens strain A with bacillaene | 19.87 |
| cluster | |
| B. amyloliquefaciens strain A baeJ knockout | 9 |
| strain | |
| B. subtilis strain B (without bacillaene cluster) | 9 |

The data show that the B. amyloliquefaciens strains which produce bacillaene inhibit the growth of S. enterica subsp. enterica enteritidis DSM 14221 whereas the baeJ knockout mutant and the B. subtilis strain which do not produce bacillaene do not inhibit the pathogen.

TABLE 2
Comparison of B. amyloliquefaciens strains encoding for the bacillaene cluster, a baeJ
knockout mutant and a B. subtilis strain not harboring the bacillaene cluster in its genome
regarding the inhibitory capacity on a pathogenic E. coli ATCC 11775 strain in a well diffusion
antagonism assays on TSBYE medium, values in mm clearance of pathogen

| Pathogen → | E. coli ATCC 11775 |
|---|---|
| strain ↓ | [mm] |
| B. amyloliquefaciens DSM 33014 | 17.36 |
| B. amyloliquefaciens strain A with bacillaene | 14.61 |
| cluster | |
| B. amyloliquefaciens strain A baeJ knockout | 9 |
| strain | |
| B. subtilis strain B (without bacillaene cluster) | 9 |

The data show that B. amyloliquefaciens strains which produce bacillaene inhibit the growth of E. coli ATCC 11775 whereas the baeJ knockout mutant and the B. subtilis strain which do not produce bacillaene do not inhibit the pathogen.

TABLE 3
Comparison of B. amyloliquefaciens strains encoding for the bacillaene cluster and a baeJ
knockout mutant regarding the inhibitory capacity on a pathogenic V. parahaemolyticus DSM
10027 strain in a well diffusion antagonism assays on TSBYE medium, values in mm clearance of
pathogen.

| Pathogen → | Vibrio parahaemolyticus |
|---|---|
| | DSM 10027 |
| strain ↓ | [mm] |
| B. amyloliquefaciens DSM 33014 | 12.90 |
| B. amyloliquefaciens strain A with bacillaene | 14.32 |
| cluster | |
| B. amyloliquefaciens strain A baeJ knockout | 9 |
| strain | |

The data show that B. amyloliquefaciens strains which produce bacillaene inhibit the growth of V. parahaemolyticus DSM 10027 whereas the baeJ knockout mutant which does not produce bacillaene does not inhibit the pathogen.

TABLE 4
Comparison of a B. amyloliquefaciens strain encoding for the bacillaene cluster and the
respective baeJ knockout mutant regarding the inhibitory capacity on C. perfringens ATCC 13124
in a well diffusion antagonism assays on TSBYE medium, values in mm clearance of pathogen.

| Pathogen → | C. perfringens ATCC 13124 |
|---|---|
| strain ↓ | [mm] |
| B. amyloliquefaciens strain A with bacillaene | 24.7 |
| cluster | |
| B. amyloliquefaciens strain A baeJ knockout | 14.75 |
| strain | |

The data show that B. amyloliquefaciens strains which produce bacillaene inhibit the growth of C. perfringens ATCC 13124 whereas for the baeJ knockout mutant which does not produce bacillaene a reduced inhibition against C. perfringens ATCC 13124 is observed.

Inhibition of C. perfringens ATCC 13124 by B. amyloliquefaciens DSM 33014 was analyzed in liquid milieu. B. amyloliquefaciens DSM 33014 was grown overnight in 10 mL of LB Kelly medium. The culture was centrifuged and filtered over a 0.2 micron cellulose acetate membrane to remove cells and spores. The cell and spore free supernatant was mixed 1:10 with double strength brain-heart-infusion (GranuCult™ BHI broth, Merck). As negative control LB Kelly was used instead of the cell and spore free supernatant. C. perfringens ATCC 13124 was grown under anaerobic conditions at 37° C. overnight in 2×BHI and inoculated 1:10 into the supernatant/2×BHI mixture and negative control at a 1:100 dilution. Upon overnight growth at 37° C. the culture of C. perfringens ATCC 13124 showed no growth (clear medium) in the presence of the cell and spore free supernatant of B. amyloliquefaciens DSM 33014, but abundant growth in the negative control (turbid medium). Thus, B. amyloliquefaciens DSM 33014 encoding the bacillaene cluster is able to inhibit C. perfringens ATCC 13124 in liquid.

### Literature

Parente, E., Brienza, C., Moles, M., & Ricciardi, A. 1995: A comparison of methods for the measurement of bacteriocin activity. Journal of microbiological methods, 22(1), 95-108.
Chen, X.-H., Vater, J., Piel, J., Franke, P., Scholz, R., Schneider, K., Koumoutsi, A., Hitzeroth, G., Grammel, N., Strittmatter, A. W., Gottschalk, G., Süssmuth, R. and Borriss, R. 2006: Structural and Functional Characterization of Three Polyketide Synthase Gene Clusters in Bacillus amyloliquefaciens FZB 42. Journal of Bacteriology, 188(11): 4024-4036.

### Example 2

### Strain Characteristics Relevant to Survival in the Gastrointestinal Tract

B. amyloliquefaciens DSM 33014 was screened to withstand various environmental and gut related conditions to be able to reach its full potential in the intestine of the target animal, characteristics for a superior strain as animal direct-fed microbial/probiotic.

B. amyloliquefaciens DSM 33014 was grown over night in Difco sporulation medium to generate spores (DSM, composition per Liter: 8 g bacto nutrient broth, 10 ml 10% (w/v) KCl, 10 ml 1.2% (w/v) MgSO4.7H2O and 1 ml 1 M Ca(NO3)2, 1 ml 0.01 M MnCl2, 1 ml 1 mM FeSO4) at 37° C. (Monteiro et al., 2005). This solution was used to assess spore heat stability to determine pelleting stability by exposing spores to 80° C. for 10 min (Leuschner and Bew, 2003) and bile resistance. The sample of the spore suspension was spotted on VIB plates (Difco™ Veal Infusion Broth, BD) pH 7. Plates were incubated at 37° C. and were analyzed for outgrowth of the spores.

In addition, spore survival and qualitative assessment of bile resistance of vegetative cells was analyzed. 3 μl of the heat-treated spore solution were spotted onto VIB plates containing 0.3% chicken bile or 0.3% porcine bile (Sigma). Plates were incubated overnight at 37° C. and analyzed for growth of the strain.

Spores of strain DSM 33014 were viable after heat treatment of 80° C. for 10 min. In addition, strain DSM 33014 was able to grow in presence of 0.3% chicken bile or 0.3% porcine bile.

### Literature

Monteiro, S. M., Clemente, J. J., Henriques, A. O., Gomes, R. J., Carrondo, M. J. and Cunha, A. E. 2005: A procedure for high-yield spore production by Bacillus subtilis. Biotechnology Progress, 21(4):1026-31.
Scholz, R., Molohon, K. J., Nachtigall, J., Vater, J., Markley, A. L., Süssmuth, R. D., Mitchell, D. A. and Borriss, R. 2011: Plantazolicin, a novel microcin B17/streptolysin S-like natural product from Bacillus amyloliquefaciens FZB42. Journal of Bacteriology, 193(1):215-24.
Leuschner, R. G. K. and Bew, J. 2003: Enumeration of Probiotic Bacilli Spores in Animal Feed: Interlaboratory Study. Journal of AOAC International, 86(3):568-75.

1. A composition containing bacillaene or a derivative thereof.

2. The composition according to claim 1, wherein the composition contains a bacillaene producing microorganism, and wherein the bacillaene producing microorganism belongs to a genus selected from the group consisting of Bacillus, Paenibacillus, and mixtures thereof.

3. The composition according to claim 1, wherein the composition is a feed composition, a food composition, a therapeutic composition, or a composition for treating plants.

4. The composition according to claim 1, wherein the composition comprises at least one feed or food ingredient selected from the group consisting of proteins, carbohydrates, fats, probiotics, probiotics, enzymes, vitamins, immune modulators, milk replacers, minerals, amino acids, coccidiostats, acid-based products, and medicines.

5. A method for treating a disease in an animal or human related to bacterial infection by Clostridia, Salmonella, and/or Vibrio, the method comprising:
administering the composition according to claim 1 to the animal or human.

6. A bacillaene producing microorganism, wherein the bacillaene producing microorganism grows in presence of 0.3 wt.-% bile.

7. The bacillaene producing microorganism according to claim 6, wherein the bacillaene producing microorganism inhibits growth of C. perfringens, Salmonella enterica, E. coli, and/or Vibrio parahaemolyticus.

8. The bacillaene producing microorganism according to claim 6, wherein the bacillaene producing microorganism grows anaerobically and/or wherein at least 50% of spores of the bacillaene producing microorganism survive exposure to 99° C. for 20 minutes.

9. The bacillaene producing microorganism according to claim 6, wherein the bacillaene producing microorganism belongs to a genus selected from the group consisting of Bacillus and Paenibacillus.

10. A method of feeding animals, comprising:
administering to the animals the bacillaene producing microorganism according to claim 6 or a preparation thereof, or a feed composition containing the bacillaene producing microorganism or the preparation thereof.

11. A method of improving the health status of an animal or a human being, comprising:
administering to the animal or human being the bacillaene producing microorganism according to claim 6 or a preparation thereof, or a composition containing the bacillaene producing microorganism or the preparation thereof, and/or bacillaene or a derivative thereof.

12. A method of improving the general physical condition of animals and/or of improving feed conversion rate of animals and/or of decreasing mortality rate of animals and/or of increasing survival rates of animals and/or of improving weight gain of animals and/or of increasing disease resistance of animals and/or of increasing immune response of animals and/or of establishing or maintaining a healthy gut microflora in animals and/or of reducing pathogen shedding through feces of animals, the method comprising:
administering to the animals at least one bacillaene producing microorganism according to claim 6 or a preparation thereof, or a composition containing the bacillaene producing microorganism or the preparation thereof.

13. A method of controlling and/or avoiding detrimental environmental effects of manure or contaminated liquids, the method comprising:
applying to the manure, the contaminated liquids, litter, a pit, or a manure pond at least one bacillaene producing microorganism according to claim 6 or a preparation thereof, or a composition containing the bacillaene producing microorganism or the preparation thereof.

14. A method of controlling and/or improving the quality of water or an aqueous solution, the method comprising:
applying to the water or aqueous solution at least one bacillaene producing microorganism according to claim 6 or a preparation thereof, or a composition containing the bacillaene producing microorganism or the preparation thereof.

15. A method of treating and/or preventing a microbial disease of a cultivated plant, comprising:
applying to the cultivated plant at least one bacillaene producing microorganism according to claim 6 or a preparation thereof, or a composition containing the bacillaene producing microorganism or the preparation thereof.

16. The bacillaene producing microorganism according to claim 6, Wherein the bacillaene producing microorganism grows in presence of 0.3 wt.-% chicken bile and/or 0.3 wt.-% porcine bile.

17. The bacillaene producing microorganism according to claim 6, wherein the bacillaene producing microorganism survives exposure to 0.3 wt.-% bile for at least 3 hours.

18. The bacillaene producing microorganism according to claim 7, wherein the C. perfringens is C. perfringens ATCC 13124;
wherein the Salmonella enterica is Salmonella enterica subsp. enterica enteritidis DSM 14221;
wherein the E. coli is E. coli ATCC11775; and/or
wherein the Vibrio parahaemolyticus is Vibrio parahaemolyticus DSM 10027.

19. The bacillaene producing microorganism according to claim 8, wherein the bacillaene producing microorganism degrades water-insoluble cellulose and protein under anaerobic conditions.

20. The bacillaene producing microorganism according to claim 9,
wherein the genus is Bacillus, and the bacillaene producing microorganism is selected from the group consisting of B. subtilis, B. amyloliquefaciens, B. methylotrophicus, and B. atropheus; or
wherein the genus is Paenibacillus, and the bacillaene producing microorganism is selected from the group consisting of P. polymixa and P. durus.
